(19) United States
(12) Patent Application Publication
(10) Pub. No.: US 2021/0268128 A1
(43) Pub. Date: Sep. 2, 2021

(54) FLUORESCENT NANOPARTICLES AND IMAGING USES

(71) Applicant: The Board of Trustees of the Leland Stanford Junior University, Stanford, CA (US)

(72) Inventor: Stefan Harmsen, Menlo Park, CA (US)

(21) Appl. No.: 17/253,959

(22) PCT Filed: Jul. 9, 2019
(86) PCT No.: PCT/US2019/041040

§ 371 Date: Dec. 18, 2020

Related U.S. Application Data
(60) Provisional application No. 62/696,113, filed on Jul. 10, 2018.

(51) Int. Cl. A61K 49/00

(52) U.S. Cl. CPC A61K 49/0093; A61K 49/005

(57) ABSTRACT

Biodegradable fluorescent silica nanoparticle (FSN) are provided for in vivo imaging, particularly of cancerous and precancerous lesions in the gastrointestinal tract. The FSN are comprised of (a) a dye that fluoresces in the near infrared spectrum which is (i) covalently joined to a silane, and (ii) distributed throughout the nanoparticle; and (b) silica distributed throughout the nanoparticle. The surface may be coated with hydroxyl-terminated PEG, which is shown to reduce uptake of the nanoparticles by the liver. The dyes provide for sensitive detection of clinically relevant lesions, and are biodegradable.

### CROSS REFERENCE

This application claims the benefit and is a 371 Application of PCT Application No. PCT/US2019/041040, filed Jul. 9, 2019, which claims benefit of U.S. Provisional Application No. 62/696,113, filed Jul. 10, 2018, which are incorporated herein by reference in their entirety.

### STATEMENT OF GOVERNMENT SUPPORT

This invention was made with Government support under contracts CA199075 and CA182043 awarded by the National Institutes of Health, and under contract 1542152 awarded by the National Science Foundation. The Government has certain rights in the invention.

### BACKGROUND

Medical endoscopes have been widely used in both diagnostic and surgical procedures. A promising technique for detecting a lesion in a living body during endoscopic procedures involves near infrared (NIR) fluorescence imaging, in which NIR light is used to illuminates tissue, exogenously applied fluorophores in the tissue emit fluorescence, and an imaging system captures a fluorescent image. In addition to fluorescence imaging, normal diagnostic and surgical procedures utilize endoscopy with conventional visible light imaging. Light in the red and near-infrared (NIR) range (600-1200 nm) is used to maximize tissue penetration and minimize absorption from natural biological absorbers such as hemoglobin and water. (Wyatt, Phil. Trans. R. Soc. London B 352:701-706, 1997; Tromberg, et al., Phil. Trans. R. Soc. London B 352:661-667, 1997). Also of interest, fluorescence imaging in the second near-infrared window (NIR-II), 1000-2500 nm, allows visualization of deep anatomical features with an unprecedented degree of clarity.

Besides being non-invasive, optical imaging methods offer a number of advantages over other imaging methods: they provide generally high sensitivity, do not require exposure of test subjects or lab personnel to ionizing radiation, can allow for simultaneous use of multiple, distinguishable probes (important in molecular imaging), and offer high temporal and spatial resolution (important in functional imaging and in vivo microscopy, respectively).

In high-risk patients who already undergo periodic white light endoscopic surveillance, it is estimated that about three times more dysplastic lesions; the most clinically relevant marker for malignant progression; are missed relative to healthy individuals. Particularly in patients with Barrett's esophagus (BE) or inflammatory bowel disease (IBD), this miss rate is higher because in these patients such lesions often appear subtle, nonpolypoid (flat or depressed), or are not endoscopically identifiable altogether. This significantly increases the risk of cancer and its associated mortality.

Further, endoscopic assessment and diagnosis of GI tract lesions is operator-dependent and prone to subjectivity, which increases inter-observer variability and thus further compromises diagnostic accuracy. Lastly, surveillance for gastric and colorectal lesions can be challenging due to the large surface area that needs to be surveyed, poor preparation and lack of time for the procedure. Moreover, even when disease is detected, it is often difficult to determine the true extent of the lesion, thus hampering the ability to achieve complete minimally invasive (endo-) therapeutic intervention through resection or ablation. Consequently, approximately 33% of GI tract lesions recur at or near the therapeutic site, commonly requiring more aggressive yet often non-curative (systemic) treatments that also negatively impact the patients' quality of life.

Chromoendoscopy, in which intravital dyes are applied intraluminally to enhance macroscopic structural features and provide negative contrast (i.e. the healthy tissues surrounding the lesions are stained), has shown improvement in the detection of such lesions. However, due to the perceived hassle, cost, time associated with dye administration, washing, and prolonged examination times relative to WLE, chromoendoscopy is not embraced by endoscopists and digital (image-enhanced) chromoendoscopy, such as narrow-band imaging (NBI), iScan, Fuji Intelligent ChromoEndoscopy (FICE), etc. so far has only shown marginal improvement.

Since early detection and adequate removal of (pre)malignant lesions is critical for prognosis, the problem of failed detection should be addressed by innovative imaging strategies that positively highlight such lesions and markedly improve detection during endoscopic surveillance and management in high risk patients. There is an unmet need for novel imaging approaches that reliably enable highly sensitive detection of (pre)malignant GI tract lesions.

### SUMMARY

Compositions and methods are provided for fluorescence imaging, particularly imaging of the gastrointestinal tract for cancerous and pre-cancerous lesions, which may be used, without limitation, for imaging and for guidance in endoscopic surveillance sampling. In some embodiments imaging is in the near-infrared spectrum. The subject may be a vertebrate animal, for example, a mammal, including a human.

Provided are biodegradable, fluorescent-dye embedded silica nanoparticles (FSN) useful for this purpose. The FSN of the invention are comprised of dye-conjugated silica, which is distributed throughout the particle, i.e. it is integral to the particle itself. In some embodiments the core of the FSN consists of dye-conjugated silica; or may be admixed with silica not conjugated to dye. In some embodiments the core nanoparticle is conjugated to hydroxy-terminated polyethylene glycol, which reduces liver uptake of the nanoparticles after administration. In some embodiments the dye is conjugated to silica through labile bonds, to increase biodegradation rates. The full biodegradability of the FSN provides a benefit over conventional nanoparticle-based contrast agents that are sequestered by the liver and spleen for long periods of time.

Biodegradable fluorescent silica nanoparticles are comprised or consist essentially of a fluorescent dye-conjugated silica, optionally admixed with non-dye-conjugated silica. The proportion of dye to silica can be can be varied to achieve an optimum in fluorescence emission. In some embodiments the FSN comprises a coating of hydroxy-terminated PEG. The FSN core, i.e. the dye and silica nanoparticle, has a diameter of from about 25 to about 200 nm, and may be at least about 25 nm, at least about 30 nm, at least about 50 nm, and not more than about 200 nm, not more than about 150 nm, not more than about 100 nm.

In some embodiments the fluorescent dye has an emission wavelength in the near infrared, e.g. from about 700 to about 2500 nm, between about 750 to about 1400 nm, between about 700 nm to about 800 nm, which may be in the NIR I window, from about 700 nm to about 900 nm, from about 750 nm to about 900 nm; or may be in the NIR II window, from about 900 nm to about 1400 nm. In some embodiments the dye is a clinically approved dye.

In some embodiments, methods are provided for enabling identification of premalignant lesions in patients by providing positive contrast enhancement of such lesions during fluorescence endoscopy or endotherapeutic/laparoscopic intervention . The biodegradable fluorescent silica nanoparticles (FSNs) provide positive contrast-enhancement of (pre)malignant lesions during endoscopic examination of the GI tract, such as mouth, throat, esophagus, stomach, duodenum, ileum, colon, rectum and pancreas. Administration of the biodegradable FSNs enables fluorescent-guided biopsy or fluorescent-guided therapy in patients that are at increased risk of developing such lesions, which patients may include, without limitation, patients with Barrett's esophagus, familial adenomatous polyposis (FAP) patients, etc.

The FSN fully degrade over a period of about 1 to about 4 months, and can be readministered for follow-up assessment, e.g. after about 3 weeks, after about 4 weeks, after about 6 weeks, after about 2 months. Thus in some embodiments the imaging method steps can also be repeated at predetermined intervals thereby allowing for the evaluation of emitted signal containing imaging probes in a subject or sample over time. The emitted signal may take the form of an image.

In some embodiments the FSN are administered to a patient intravenously prior to imaging, where the period of time between administration and imaging is sufficient for localization of the dye in cancerous or pre-cancerous lesions, where the period of time is sufficient for tumoritropic enhanced permeability and retention effect (EPR). It is shown herein that the dye is selectively retained by malignant and premalignant lesions, allowing detection of such lesions. The presence, absence, distribution, or level of optical signal emitted by the fluorescent nanoparticle is indicative of a disease state. In some embodiments detection of the dye is performed using fluorescence endoscopy. In some embodiments, visualization of lesion is used to guide a biopsy. Fluorescence guidance during endoscopy improves diagnostic accuracy and/or therapeutic efficacy.

Also provided herein is a method of in vivo optical imaging, the method comprising (a) administering to a subject an FSN composition; (b) allowing time for the FSN to distribute within the subject or to contact or interact with a biological target; (c) illuminating the subject with light of a wavelength absorbable by the FSN; and (d) detecting the optical signal emitted by the FSN. The optical signal generated by the FSN, whether collected by tomographic, reflectance, planar, endoscopic, microscopic, surgical goggles, video imaging technologies, or other methods such as microscopy including intravital and two-photon microscopy, and whether used quantitatively or qualitatively, is also considered to be an aspect of the invention.

Another aspect of the invention features FSN formulated in a pharmaceutical composition suitable for administration to animal, including human, subjects. The pharmaceutical composition can include the nanoparticles and one or more stabilizers in a physiologically relevant carrier. In some embodiments a pharmaceutical composition is provided, comprising one or more of the FSN and a pharmaceutically acceptable excipient. In some embodiments the pharmaceutical composition is provided in a unit dose, e.g. at a dose of from about 1 fmol/g, from about 10 fmol/g, from about 25 fmol/g, from about 50 fmol/g, from about 100 fmol/g, from about 200 fmol/g, from about 500 fmol/g, from about 750 fmol/g, from about 1 pmol/g; and not more than about 100 pmol/g, not more than about 10 pmol/g.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Nanoparticle contrast agents highlight (pre)malignant lesions of the digestive tract, showing clinical use of biodegradable fluorescent nanoparticles. While (pre)malignant lesions are difficult to detect using conventional white light endoscopy. Intravenously administered nanoparticle-based optical contrast agents highlight dysplastic lesions along the entire digestive tract to improve the detection of these clinically relevant lesions and allow therapeutic intervention to prevent malignant transformation.

FIG. 2A-2G. FSN synthesis and characterization. FIG. 6A-6C. a NIRF dye was appended to a silane and covalently incorporated to yield the FSNs, which were further functionalized with MPTMS to enable straightforward PEGylation using maleimide chemistry. FIG. 6D. Optimal NIRF-silane concentration (in μM) during synthesis that results in high NIRF intensity (at equimolar concentration of FSNs) was found to be 0.4-0.8 μM. FIG. 6E-F. FSN size was 101±18 nm. FIG. 6G. the detection limit of FSNs was 10×10⁻¹⁵ M.

FIG. 3A-3B. Biodegradable, near-infrared fluorescent nanoparticle characterization and tumor uptake mechanism. FIG. 3A. relative to the vasculature of normal tissues, vascular changes associated with tumor progression facilitate the extravasation, passive accumulation and retention of intravenously administered nanoparticles at the tumor site. This phenomenon is known as the enhanced permeability and retention effect (EPR). FIG. 3B. Table 1, properties of optical imaging agents.

FIG. 4A-4C. FSNs delineate intestinal tumors. FIG. 4A. Wide-field NIRF imaging of the intestine of an Apc^{Min/+} mouse intravenously injected with FSNs enabled detection of lesions (in red). FIG. 4B. Histopathology identified the lesions on the corresponding H&E-stained tissue section as adenomas. FIG. 4C. H&E staining did not compromise the fluorescence emission of the FSNs. FSN fluorescence emission colocalized with the basophilic (H&E) tumor stain (right panel; TBR=10).

FIG. 5A-5C. Schematic of the NIRF endoscopy system that was used to perform the NIRF endoscopy in the rats. FIG. 5A. 660-nm laser (IBeamSmart PT, Toptica Photonics AG, Gräfelfing, Germany) was coupled via a beam separation block (BSM) using a FLO to a Spyglass fiberscope (Boston Scientific, Marlborough, Mass.), which has an outer diameter of ˜0.9 mm and contains 225 multimode illumination fibers, 6600 collection fibers, and a 400-μm diameter lens (μFL), and provides a 70o field of view (FOV). Laser power output was measured to be 10 mW at the distal end. The collection fibers are focused (FL1: 20′ Infinity-corrected Achromat Objective, F=9.0 mm, NA=0.4, WD=1.2 mm, Thorlabs # RMS20′; FL2: Infinity-corrected Tube Lens, Thorlabs # ITL200), filtered (LP: Long wavelength-pass filter 664 nm RazorEdge, LP02-664RU-25), and dispersed onto a 1-MPixel EMCCD camera (Luca R, Andor Technology, Belfast, UK). FIG. 5B, Photo of the custom-built NIRF/WL endoscopy system. The Spyglass fiberscope was fed into the instrument channel of a clinical white-light endoscopy system (EPK-1000, Pentax Medical, Montvale, N.J.). The white-light endoscopy was recorded using GrabBee software and NIRF imaging was recorded using Solis software (Andor Technology). The combined WL/NIRF imaging was recorded using screen capturing software (Captivate, Adobe Inc, San Jose, Calif.). FIG. 5C, Schematic of the combined NIRF/WL endoscopy system that was used to perform the combined NIRF/WL endoscopy in the pigs. In brief, the imaging setup was designed to offer video-rate simultaneous color and near-infrared (NIR) fluorescence endoscopy. The system employs a multipurpose imaging fiber bundle with 30,000 coherently arranged individual fibers (viZaar, Albstadt, Germany) achieving a 70° front viewing field of view. White-light illumination for color imaging was provided by a 250-W halogen lamp (KL-2500 LCD, Schott AG, Mainz, Germany), filtered by a 665 nm short-pass filter (FF01-665/SP-25, Semrock, Rochester, N.Y., United States) and NIRF excitation was achieved by a laser diode emitting at 670 nm (SLD1332V, Thorlabs, Newton, N.J., United States). A multibranched fiber optic bundle (Leoni FiberOptics, Neuhaus-Schierschnitz, Germany) realized simultaneous white-light illumination and NIRF excitation coupling to the flexible fiberscope. The images propagated through the fiberscope were relayed by a NIR achromatic doublet pair (RL: MAP10100100-B, Thorlabs) and separated by a dichroic mirror (DM: FF685-Di02, Semrock) into color and fluorescence channels. The color channel was filtered by a 665 nm short-pass filter (SP: FF01-665/SP-25, Semrock) for both cases and recorded by a 12-bit color charge-coupled device (CCD) camera (Pixelfly qe, PCO AG, Kelheim, Germany). NIRF detection spectral bandwidth was defined by a 685-nm long-pass filter (LP: FF01-685/LP-25, Semrock) followed by a 732/68-nm band-pass filter (BPF: FF01-732/68-25, Semrock) and recorded by an iXon electron multiplying charge-coupled device (EMCCD, DV897DCS-BV, Andor Technology, Belfast, Northern Ireland). The power at the distal end of the endoscope complied with the American National Standards Institute (ANSI) and the European Standards (EN) limits for the maximum permissive exposure in skin (19 mW/cm² for the 670-nm laser measured at distance <2 mm).

FIG. 6A-6C. FSN-augmented NIRF/WL endoscopy in Apc^{Pirc/+}. FIG. 6A. In a study with the FSNs, a protruding polyp that was identified by WLE is visualized with high sensitivity using NIRF endoscopy. FIG. 6B. Ex vivo wide-field NIRF imaging on the open colon corroborated the presence of polyps and re-emphasized the high sensitivity (TBR>10) of FSN-augmented NIRF (endoscopic) imaging. FIG. 6C. H&E and confocal NIRF microscopy of polyp 1 demonstrated that the FSNs (II; arrow heads) selectively co-localize with the basophilic H&E stain (dysplastic tissue (D); II) and are not associated with normal or hyperplastic tissue (H; I). Scale bars, 50 μM.

FIG. 7A-7B. Effect of surface chemistry on protein corona formation and FSN biodistribution. FIG. 7A. Non-conjugated (SH), methoxy-PEG_{2000da} (mPEG)-, and hydroxy-PEG_{2000da}-conjugated FSNs were incubated in human serum for 1 h at 37° C. After incubation, the FSNs were washed and FSNs were submitted to SDS-PAGE under denaturing conditions. The proteins were stained with coomassie-blue staining. Relative to bare (SH) and mPEG-, PEG-OH conjugation reduced total protein adsorption to the FSNs. FIG. 7B, Equimolar doses of mPEG- or PEG-OH-conjugated FSNs were injected intravenously in Apc^{Min/+} mice. Wide-field NIRF imaging revealed that PEG-OH reduced the hepatic uptake of FSNs. Arrow head pinpoints the presence of an intestinal polyp.

FIG. 8A-8B. Biodegradability of FSNs. FIG. 8A. NIRF imaging of hepatic uptake and clearance of FSNs in nude mice (in months post-injection; t=0 is 1 day post injection). FIG. 8B. Following intravenous injection of the first-generation FSNs (30 fmol/g), the hepatic fluorescence signal decreases in the weeks post intravenous. After 4 months, the hepatic fluorescence signal is no longer significantly different from the background (dotted line; *P<0.05). Of note: we are awaiting TEM of the liver.

FIG. 9A-9E. Gastrointestinal tumor accumulation of FSNs in Apc^{Min/+} mice. 9A, Near-infrared (NIR) fluorescence imaging (lex 680 nm; lem >700 nm) was performed on freshly resected ileal tissues of Apc^{Min/+} mice that had received FSNs (i.v. 30 fmol/g; n=5). Tumor-to-background ratios (TBR) were 4.0, 3.6, 3.0 for polyps 1-3, respectively. Fluorescence intensity is displayed in arbitrary units. Scale bar, 10 mm. 9B, H&E stained tissue section (10-μm). Scale bar, 10 mm. 9C, 4× magnification and NIR fluorescence imaging of the selected area (panel 9A and 9B) showing that the fluorescence signal of the FSNs aligns well with polyps 1-3. Scale bar, 2.5 mm. 9D, 20× magnification NIRF microscopy imaging (lex 680 nm; lem>700 nm) of the polyp in panel 9C demonstrates the fluorescent nanoparticles mainly localized to the tumor stroma (‘s’) and not to epithelial cells (‘e’). Inset is a 4× higher magnification of the indicated area. 9E, Fluorescence-activated cell sorting (FACS) analysis of the polyps of Apc^{Min/+} mice (n=2) showed that the mean fluorescence intensity (MFI) of the FSNs was mainly associated with macrophages and neutrophils.

FIG. 10A-10D. FSN-augmented combined NIRF/WL endoscopy in APC^{1311/+} pigs. 10A, White-light (left panel), NIRF (middle panel), and combined NIRF/WL (right panel) endoscopic images of a colorectal polyp in APC^{1311/+} pigs (n=2) 18 h after intravenous administration of FSNs (4.7 fmol/g). 10B, White-light, and 10C, wide-field NIRF imaging (lex 680 nm; lem>700 nm) of a randomly resected colorectal tissue section from the scoped APC^{1311/+} pig. Fluorescence intensity is expressed as arbitrary units. Scale bar, 10 mm. 10D, Representative H&E stained section of a NIRF-positive lesions corroborated the presence of adenomatous polyps (black arrowhead). Scale bar, 1 mm.

FIG. 11A-11B. Fluorescent nanoparticle synthesis. 11A, CF680R-maleimide was reacted with (3-mercaptopropyl)trimethoxysilane (MPTMS) in a 1:2 molar ratio in N,N-dimethylformamide (DMF) to yield CF680R-MPTMS. 11B, Fluorescent silica nanoparticles were synthesized by reacting the silane-appended dye (concentration range CF680R-MPTMS: 0.19-3 μM, the silica precursor tetraethyl orthosilicate (TEOS; 4.5% (v/v)), 0.7% (v/v) ammonium hydroxide, and 8% (v/v) water in 2-propanol. Reaction conditions: i, 15 min, ambient conditions; ii, 5.5% (v/v) MPTMS, 0.5% (v/v) ammonium hydroxide in ethanol, while shaking (350 rpm) for 2-h at ambient conditions; iii, Excess maleimide-functionalized hydroxy-terminated polyethyleneglycol (PEG-OH; 3.4 kDa) in 10 mM 3-(N-morpholino)propanesulfonic acid (MOPS; pH 7.3), while shaking (350 rpm) for at least 2-h at ambient conditions.

FIG. 12. Reproducibility. Five different batches of FSNs were produced and NIRF of each batch was measured at 1 nM in EtOH (lex,em=680 nm, >700 nm). Coefficient of variation is 0.9%. Fluorescence intensity is expressed as arbitrary units. Error bars represent standard deviation

FIG. 13. In vivo biodegradation of FSNs. Transmission electron microscopy (TEM) image of a spleen of the nude mouse that was sacrificed at 2-months post injection of FSN (30 fmol/g). At this time-point, the different stages of FSN degradation (etching from the inside, collapse, and dissolution; arrow heads) were fully captured in situ. Scale bar, 500 nm.

FIG. 14. Photostability. The photostability of a standard concentration series (range 0.03-300 pM) of FSNs that was included in the biodegradation study. As such, the standard was imaged over the course of 6 months and subjected to ˜200 exposures (1 s exposure time) during imaging on a Pearl Trilogy (Li-Cor). Coefficient of variations (CV) were less than 5% for all concentrations. Fluorescence intensity is expressed as arbitrary units.

FIG. 15. Histological assessment of organs following long-term FSN exposure. H&E sections of the mononuclear phagocyte system (MPS) organs (liver, spleen, and bone marrow) and kidneys following 6-months post tail-vein injection of FSNs (30 fmol/g) in nude mice (n=5). The organs of mice intravenously administered with non-PEGylated FSNs (30 fmol/g) or FSNs (30 fmol/g), were histologically indistinguishable from the vehicle (5% D-glucose; D5W) controls. No histologic abnormalities were noted in the liver (CV: central vein; PT: portal triad; H: hepatocytes), spleen (RP: red pulp; T: trabecula; WP: white pulp), kidney (C: cortex; M: medulla), or femoral bone marrow (B: femoral bone; BM: femoral bone marrow). Scale bar, 100 μm.

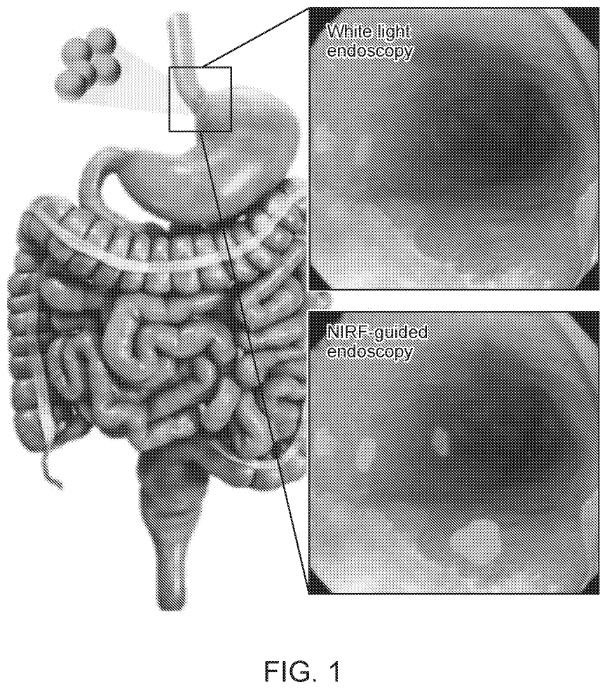

### DETAILED DESCRIPTION OF THE EMBODIMENTS

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a fluorescent nanoparticle” includes a plurality of such fluorescent nanoparticles known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions of other terms and concepts appear throughout the detailed description below.

The terms “individual,” “subject,” “host,” and “patient,” used interchangeably herein, refer to an individual organism, e.g., a mammal, including, but not limited to, murines, simians, non-human primates, humans, mammalian farm animals, mammalian sport animals, and mammalian pets.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

Fluorescent dyes. The present invention utilizes bright, highly fluorescent compounds (dyes) that absorb and/or emit in the near infrared spectrum, between about 700 to about 2500 nm, between about 750 to about 1400 nm, between about 700 nm to about 800 nm, which may be in the NIR I window, from about 700 nm to about 900 nm, from about 750 nm to about 900 nm; or may be in the NIR II window, from about 900 nm to about 1400 nm.

Fluorescent dyes of interest include without limitation polymethines, cyanines, rhodamine analogs, 4,4-difluoro-4-bora-3a,4a-diaza-s-indacene (BODIPYs), squaraines, chalcogenopyrylium, flavylium polymethines, (na)phthalocyanines, and porphyrin derivatives and other related dyes, including for example indocyanine green, heptamethine carbocyanine IR-783 and its derivative MHI-148 as well as fluorescent hyaluronan (HA) analogs linking different molar percentages of IR-783 derivative; NIRF heptamethine dyes, IR780 and IR808; (PEG)ylated IR-786 derivative; octupolar merocyanine chromophores; 1,3-bis(dicyanomethylidene)indan; rhodamine derivatives such as SiR680 and SiR700; 2-Me TeR; BODIPY derivatives such as diphenyl dithienyl aza-BODIPY, bromo-substituted BODIPY containing thienopyrrole moieties, DC-SPC; DC-SPC-PPh3, Squaraines (squarylium dyes), phthalocyanines and porphyrin derivatives including hydrophilic porphyrin (THPP) and its derivative (Zn-THPP, zwitterionic NIR fluorophore ZW800-1; 2′,7′-dichlorofluorescein. In some embodiments the dye is a clinically approved dye. In some embodiments the dye is one or more of ICG, IRdye800CW, IR783, IR780 or S0456 (CAS # 1252007-83-2).

Fluorescent silica nanoparticles. The fluorescent-dye embedded silica nanoparticles (FSN) of the invention are comprised of dye-conjugated silica, which is distributed throughout the particle, i.e. it is integral to the particle itself. In some embodiments the core of the FSN consists of dye-conjugated silica; or may be admixed with silica not conjugated to dye. Biodegradable fluorescent silica nanoparticles therefore may be comprised of, or consist essentially of, a fluorescent dye-conjugated silica, optionally admixed with non-dye-conjugated silica.

The selected dye or combination of dyes, e.g. 2, 3, 4 or more dyes, are chemically bound to a silane molecule prior to formation of the nanoparticle. The selected chemistry is adjusted based on the nature of the dye. The conjugation may be through a linker, or may directly join the dye to silane. Exemplary chemistries include, without limitation, maleimide or NHS-functionalized dyes. The dyes are conjugated to, for example, a modified silane such as 3-mercaptopropyltrimethoxysi lane (MPTMS), 3-mercaptopropyltriethoxysilane (MPTES), 3-aminopropyltrimethoxysilane (APTMS), 3-aminopropyltriethoxysilane (APTES), etc. Alternatively a chloro-substituted dye, e.g. mesochloro-substituted cyanine., e.g. IR780 iodide, IR783, etc., benz[cd]indolium (e.g. IR1048, etc.), or pyrylium (e.g. IR1061, etc.) dye are conjugated to triethylamine (TEA) or diisopropylethylamine (DIEA) silanes.

Nanoparticles are formed by reacting silane conjugated dyes with a silica precursor, e.g. tetraethyl orthosilica (TEOS), methyltriethoxysilane (MTES), γ-aminopropylsilanetriol, (APSTOL), etc.

Optionally a fraction of the silica precursor comprises a labile-bond, e.g. (disulfide, ester, cleavable peptide , etc.), which may be present or not present, e.g. at a concentration of 0%, from about 1%, about 5%, about 10%, up to about 50%, up to about 40%, up to about 30%, up to about 25% of the silica precursor.

Addition of labile bonds can be used to decrease the time for the particles to biodegrade after injection. The time for complete biodegradation of the FSN following injection may be up to about 6 months, up to about 5 months, up to about 4 months, up to about 3 months, or less. The half-life kinetics, however, allow the level of detectable FSN to drop significantly in the first 4 weeks, first 6 weeks, first 8 weeks, first 12 weeks, etc., following injection, and thereby allow repeated screening with a second dose of FSN, after such a period of time.

The proportion of dye to silica can be can be varied to achieve an optimum in fluorescence emission. For example the dye-conjugated silica may be present at a ratio of from about 1:100 with unconjugated silica, from about 50:1, from about 25:1, from about 10:1, about 5:1, about 2:1, about 1:1, about 1:2, about 1:5, up to about 1:10, up to about 1:25, up to about 1:50, up to about 1:100. The proportions can be optimized for brightness.

The size of the nanoparticles can be controlled during the process of aggregating the silica molecules, e.g. by adjusting the solvents during particle formation. Preferably the nanoparticles are at least about 10 nm in diameter and not more than about 250 nm in diameter, more usually at least about 50 nm in diameter and not more than about 150 nm in diameter, and may be from about 75 nm in diameter to from about 125 nm in diameter.

The FSN core, i.e. the dye and silica nanoparticle, may have a diameter of from about 25 to about 200 nm, and may be at least about 25 nm, at least about 30 nm, at least about 50 nm, and not more than about 200 nm, not more than about 150 nm, not more than about 100 nm.

In some embodiments the fluorescent dye has a wavelength in the near infrared, e.g. from about 700 to about 2500 nm, between about 750 to about 1400 nm, between about 700 nm to about 800 nm, which may be in the NIR I window, from about 700 nm to about 900 nm, from about 750 nm to about 900 nm; or may be in the NIR II window, from about 900 nm to about 1400 nm.

The limit of detection may range from, but is not limited to, from about 1 femtomolar (10⁻¹⁵ M) to about 1 picomolar (10⁻¹² M) on a per particle basis, for example from 10⁻¹² M, from about 10⁻¹³ M, from about 10⁻¹² M, to about 30×10⁻¹⁵ M, to about 10⁻¹⁴ M.

The FSN may be modified on the surface to covalently attach, for example, hydroxyl-terminated PEG or targeting moieties (e.g. antibodies, etc.). Surface functionality can be introduced to the FSN by reacting with functional silanes, e.g. 3-mercaptopropyltrimethoxysilane (MPTMS), 3-aminopropyltrimethoxysilane (APTES), etc. The functionality allows conjugation to a surface coatings, for example maleimide conjugated PEG, to link the PEG to the nanoparticle through sulfhydryl functionality. Various sizes of PEG may be used. Purified PEG is commonly available commercially as mixtures of different oligomer sizes in broadly or narrowly defined molecular weight (MW) ranges. For example, the size for conjugating to FSN may have a MW from about 400 da, from about 600 da, from about 1000 da, from about 2000 da, up to about 20,000 da, up to about 15,000 da, up to about 10,000 da, up to about 5000 da, for example from about 600 da to about 5000 da.

When PEG is present, hydroxyl-terminated PEG is preferred. The PEG may be present at a concentration of from about 500 PEG polymers per particle, from about 1000, from about 5,000 from about 10,000, up to about 500,000, up to about 100,000, up to about 50,000.

The FSN is typically delivered parentally, where the term includes intravenous, intramuscular, subcutaneous, intraarterial, intraarticular, intrasynovial, intrasternal, intrathecal, intraperitoneal, intracisternal, intrahepatic, intralesional, intracranial and intralymphatic injection or infusion techniques. Alternative administration may be orally, parentally, by inhalation, topically, rectally, nasally, buccally, vaginally, or via an implanted reservoir.

The dose may depend on the brightness of the dye, and can be, for example, e.g. at a dose of from about 1 fmol/g, from about 10 fmol/g, from about 25 fmol/g, from about 50 fmol/g, from about 100 fmol/g, from about 200 fmol/g, from about 500 fmol/g, from about 750 fmol/g, from about 1 pmol/g; and not more than about 100 pmol/g, not more than about 10 pmol/g.

Imaging is performed with a laser appropriate for the dye. Excitation light in the NIR spectrum with wavelengths shorter than the fluorescent emission maximum is used to illuminate the tissue and excites the fluorophores in the tissue. The resulting fluorescent emission is detected at NIR wavelengths longer than the excitation light based on the Stokes shift. The fluorescence quantum yields give the efficiency of the fluorescence process, which is normally low. As a result, the intensity of the fluorescent emission is generally very weak compared to the intensity of the NIR excitation light. Therefore, in order to observe the fluorescence image, an optical filter is utilized to block the NIR excitation light from reaching the sensor.

A CCD, CMOS, or InGaAs image sensor typically has a spectral response from 200 nm to 1800 nm, allowing the sensor to capture light for imaging in both the visible and NIR regions. However, the spectral response of an image sensor in the NIR spectrum is only 10%-30% of its peak response in the visible spectrum.

FSN can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, the subject compound is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures. In some embodiments, the subject compound is formulated for sustained release.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

An FSN can be formulated for administration by injection. Typically, injectable compositions are prepared as liquid solutions or suspensions; solid forms suitable for solution in, or suspension in, liquid vehicles prior to injection may also be prepared. The preparation may also be emulsified or the active ingredient encapsulated in liposome vehicles.

### Methods

The FSN, and pharmaceutical compositions can be administered prior to imaging, e.g. at least about 4 hours prior to imaging, at least about 8 hours, at least about 12 hours, at least about 18 hours, and may be administered up to about 12 hours, up to about 18 hours, up to about 24 hours, up to about 36 hours, up to about 48 hours or more, provided that the time is sufficient to enable localization of the FSN at sites of lesions, e.g. dysplasia lesions. and prior to biodegradation of the FSN, e.g. not more than about 1 week prior to imaging. In one embodiment, an effective dose, which is an amount effective to generate a detectable signal of a lesion, if a lesion is present, of the FSN is administered by parenteral injection to an individual, followed by imaging of the FSN in the individual.

Since pre-malignant lesions have a high probability of progressing to (colorectal) cancer in the future, detection results obtained according to the detection method of the present invention serve as extremely useful information when assessing the risk of existing cancer, including colorectal cancer, and during minimally invasive assessment of the risk of future colorectal cancer at an early stage. For example, according to the detection method of the present invention, the subject can be assessed has having a high risk of the onset of colorectal cancer and colorectal adenoma in the future.

In some embodiments, an individual is imaged with FSN as described herein for detection of hyperproliferative conditions, including without limitation hyperproliferative conditions of the GI tract, including malignant and premalignant lesions. The term GI tract includes, for example, oral cavity, esophagus, stomach, small intestine, and large intestine. Gastrointestinal cancer refers to malignant conditions of the GI tract and accessory organs of digestion, including the esophagus, stomach, biliary system, pancreas, small intestine, large intestine, rectum and anus. The symptoms relate to the organ affected and can include obstruction (leading to difficulty swallowing or defecating), abnormal bleeding or other associated problems. The diagnosis often requires endoscopy, followed by biopsy of suspicious tissue.

Esophageal cancer is the sixth-most-common cancer in the world. There are two main types of esophageal cancer—adenocarcinoma and squamous cell carcinoma. Adenocarcinomas of the esophagus tend to arise in a field defect called Barrett's esophagus, a red patch of tissue in the generally pink lower esophagus. Esophageal squamous-cell carcinomas may occur as second primary tumors associated with head and neck cancer. Cancer of the stomach, also called gastric cancer, is the fourth-most-common type of cancer. The most common type of gastric cancer is adenocarcinoma. Pancreatic cancer is the fifth-most-common cause of cancer deaths in the United States. These cancers are classified as endocrine or nonendocrine tumors. The most common is ductal adenocarcinoma. Colorectal cancer may be associated with hereditary syndromes like Peutz-Jegher's, hereditary nonpolyposis colorectal cancer or familial adenomatous polyposis, or may be age related. Colorectal cancer can be detected through the bleeding of a polyp, colicky bowel pain, a bowel obstruction or the biopsy of a polyp at a screening colonoscopy. Anal cancers include carcinomas and squamous cell carcinomas.

Colorectal cancer (CRC) is the third most common malignant neoplasm worldwide and the second leading cause of cancer deaths in the United States. It is estimated that there will be 140,250 new cases diagnosed in the United States in 2018 and 50,630 deaths due to this disease. The major factor that increases a person's risk for CRC is increasing age. Risk increases dramatically after age 50 years with 90% of all CRCs diagnosed after this age. History of CRC in a first-degree relative, especially occurring before age 55, roughly doubles the risk. A personal history of CRC or high-risk adenomas (i.e., large [>1 cm] tubular adenomas, sessile

A benefit of the FSN in screening, including without limitation individuals at risk of colorectal cancer, is the ability to detect dysplasia. The current approach to surveillance is grounded in the concept of an inflammation-dysplasia-carcinoma sequence, with dysplasia representing a premalignant phase during which intervention can prevent or minimize the complications associated with invasive cancer. Dysplasia is defined as unequivocal neoplasia of the epithelium confined to the basement membrane, without invasion into the lamina propria. Dysplasia can be classified as raised or flat based on its endoscopic appearance. But irrespective of the endoscopic appearance of a lesion as raised or flat, pathologists use the same set of criteria to describe the histologic appearance of dysplasia. A standardized classification system divides dysplasia into categories, including indefinite dysplasia, low grade dysplasia (LGD), high grade dysplasia (HGD) and cancer. Screening with FSN may be particularly relevant for individuals with a high risk of GI tract cancer, e.g. individuals with inflammatory bowel disease (IBD), or a genetic predisposition to GI tract cancer.

Factors suggestive of a genetic contribution to CRC include: (1) a strong family history of CRC and/or polyps; (2) multiple primary cancers in a patient with CRC; (3) the existence of other cancers within the kindred consistent with known syndromes causing an inherited risk of CRC, such as endometrial cancer; and (4) early age at diagnosis of CRC.

Hereditary CRC has two well-described forms: (1) polyposis (including familial adenomatous polyposis [FAP] and attenuated FAP (AFAP), which are caused by pathogenic variants in the APC gene; and MUTYH-associated polyposis, which is caused by pathogenic variants in the MUTYH gene); and (2) Lynch syndrome (often referred to as hereditary nonpolyposis colorectal cancer), which is caused by germline pathogenic variants in DNA MMR genes (MLH1, MSH2, MSH6, and PMS2) and EPCAM. Other CRC syndromes and their associated genes include oligopolyposis (POLE, POLD1), NTHL1, juvenile polyposis syndrome (BMPR1A, SMAD4), Cowden syndrome (PTEN), and Peutz-Jeghers syndrome (STK11). Many of these syndromes are also associated with extracolonic cancers and other manifestations. Serrated polyposis syndrome, which is characterized by the appearance of hyperplastic polyps, appears to have a familial component.

Colonoscopy for CRC screening and surveillance is commonly performed in individuals with hereditary CRC syndromes and has been associated with improved survival outcomes. For example, surveillance of Lynch syndrome patients with colonoscopy every 1 to 2 years, and in one study up to 3 years, has been shown to reduce CRC incidence and mortality. Extracolonic surveillance is also a mainstay for some hereditary CRC syndromes depending on the other cancers associated with the syndrome. For example, regular endoscopic surveillance of the duodenum in FAP patients has been shown to improve survival. A benefit of imaging with FSN is improved endoscopic surveillance, where the localization of the FSN allows guidance for biopsy and imaging.

The general principles of fluorescence, optical image acquisition, and image processing can be applied in the practice of the invention. For a review of optical imaging techniques, see, e.g., Alfano et al., Ann. NY Acad. Sci. 820:248-270, 1997. An imaging system useful in the practice of methods described herein typically includes three basic components: (1) an appropriate light source for fluorescent molecule excitation, (2) a means for separating or distinguishing emissions from light used for the excitation, and (3) a detection system to detect the optical signal emitted.

In general, the optical detection system can be viewed as including an optical gathering/image forming component and an optical detection/image recording component. The optical detection system can be a single integrated device that incorporates both components.

A particularly useful optical gathering/image forming component is an endoscope. Endoscopic devices and techniques which have been used for in vivo optical imaging of numerous tissues and organs, including peritoneum, colon and rectum, bile ducts, stomach, bladder, lung, brain, esophagus, and head and neck regions can be employed in the practice of the present invention. Other types of optical gathering components useful in the invention are catheter-based devices, including fiber optics devices. Still other imaging technologies, including phased array technology, optical tomography, intravital microscopy, confocal imaging and fluorescence molecular tomography (FMT) can be employed in the practice of the present invention.

A suitable optical detection/image recording component, e.g., charge coupled device (CCD) systems or photographic film, can be used in the invention. The choice of optical detection/image recording will depend on factors including type of optical gathering/image forming component being used. Selecting suitable components, assembling them into an optical imaging system, and operating the system is within ordinary skill in the art.

### Diagnostic and Disease Applications and Methods

The methods described herein can be used to determine a number of indicia, including tracking the localization of the FSN in the subject over time, or assessing changes in the subject over time. The methods can also be used to follow therapy for such diseases by imaging molecular events and biological pathways.

The methods can be used to help a physician or surgeon to identify and characterize areas of disease, such as pre-malignant lesions, cancers and specifically colon polyps, to distinguish diseased and normal tissue, help dictate a therapeutic or surgical intervention, e.g., by determining whether a lesion is cancerous and should be removed or non-cancerous and left alone, or in surgically staging a disease. The methods can also be used in the detection, characterization and/or determination of the localization of a disease, especially early disease, the severity of a disease or a disease-associated condition, the staging of a disease, and monitoring and guiding various therapeutic interventions, such as surgical procedures, and monitoring drug therapy, including cell based therapies. The methods can therefore be used, for example, to determine the presence of tumor cells and localization of tumor cells.

The FSN and methods described herein can be used in combination with other imaging compositions and methods. For example, the methods can be used in combination with other traditional imaging modalities such as X-ray, computed tomography (CT), positron emission tomography (PET), single photon computerized tomography (SPECT), and magnetic resonance imaging (MRI). For instance, FSN can be used in combination with CT and MR imaging to obtain both anatomical and biological information simultaneously, for example, by co-registration of a tomographic image with an image generated by another imaging modality. FSN can also be used in combination with X-ray, CT, PET, SPECT and MR contrast agents or the fluorescent silicon nanoparticle imaging probes of the present invention may also contain components, such as iodine, gadolinium atoms and radioactive isotopes (Nano Letters 2015; 15(2):864-868), which can be detected using CT, PET, SPECT, and MR imaging modalities in combination with optical imaging.

### Kits

The FSN described herein can be packaged as a kit, which may optionally include instructions for using the nanoparticles in various exemplary applications. Non-limiting examples include kits that contain, e.g., the FSN in a powder or lyophilized form, and instructions for using, including reconstituting, dosage information, and storage information for in vivo and/or in vitro applications. For in vivo applications, the kit may contain FSN in a dosage and form suitable for a particular application, e.g. a liquid in a vial, etc.

The kit can include optional components that aid in the administration of the unit dose to subjects, such as vials for reconstituting powder forms, etc. The kits may be supplied in either a container which is provided with a seal which is suitable for single or multiple puncturing with a hypodermic needle (e.g. a crimped-on septum seal closure) while maintaining sterile integrity. Such containers may contain single or multiple subject doses. Additionally, the unit dose kit can contain customized components that aid in the detection of FSN in vivo or in vitro, e.g., specialized endoscopes, light filters. The kit may be manufactured as a single use unit dose for one subject, multiple uses for a particular subject, or the kit may contain multiple doses suitable for administration to multiple subjects (“bulk packaging”). The kit components may be assembled in cartons, blister packs, bottles, tubes, and the like.

### EXPERIMENTAL

### Example 1

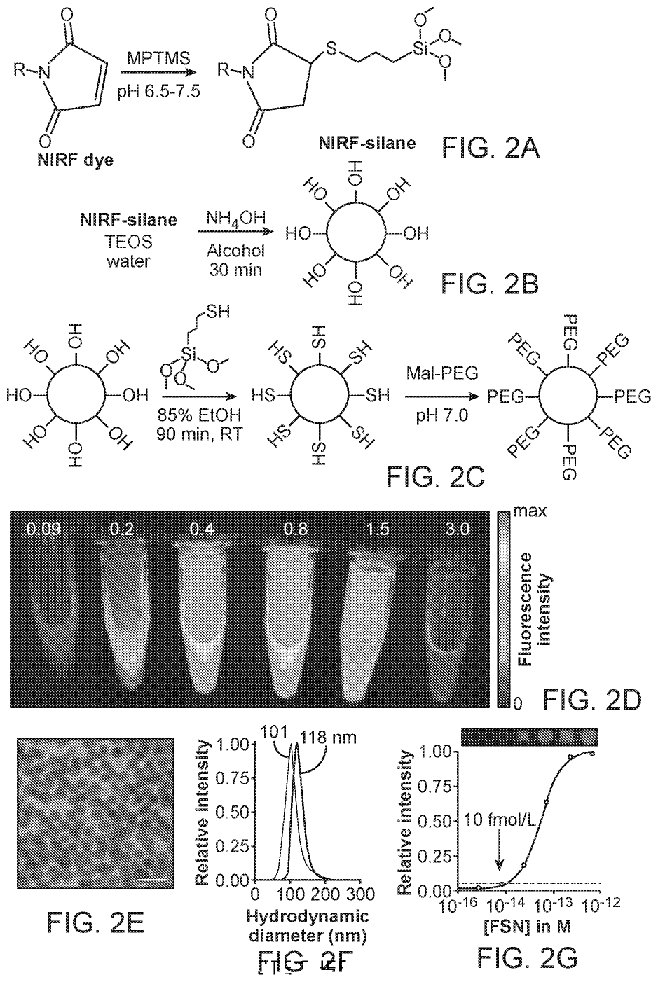

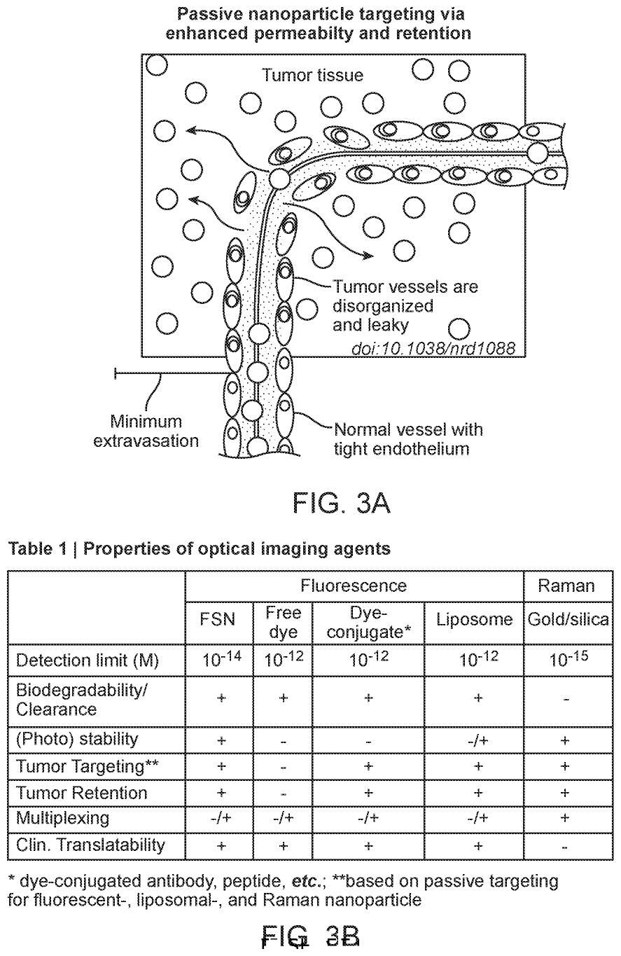

Fluorescent silica nanoparticles (FSN) have a detection limit that is only one order-of-magnitude different from Raman nanoparticles, which to date have showcased the lowest reported limit of detection using (near) real-time optical imaging. Relative to other fluorescent-based agents such as free- or targeted dyes (e.g. indocyanine green (ICG), IRdye800CW, respectively) or liposomal dye formulations, which typically have a limit of detection in the picomolar range (10⁻¹² M), FSNs have a limit of detection in the low femtomolar range (10⁻¹⁴ M; FIG. 2). Covalently-incorporated dyes within the silica matrix exhibit photophysical properties that are distinct from their solution properties, thereby leading to enhanced radiative emission and increased photostability. Unlike liposomal dye formulations, the dyes are stably bound within the silica matrix and do not leak out.

Passive tumor targeting by systemic or localized injection of dye-embedded nanoparticles, such as particles from about 10 nm to about 150 nm in diameter, obviates the need for targeting moieties, because tumor accumulation is governed by a biologically phenomenon that is shared by lesions across the cancer spectrum ranging from premalignant- to advanced malignant disease; the enhanced permeability and retention (EPR) effect (FIG. 3A). The enhanced permeability of the tumor neovasculature facilitates extravasation of nanoparticles into the tumor bed where they are locally retained due to ineffective lymphatic drainage. Since EPR strongly correlates with the degree of tumor vascularization, and dysplastic GI tract lesions commonly are highly vascularized, nanoparticles such as FSNs specifically accumulate in clinically relevant (pre)malignant lesions to enable their endoscopic detection along the entire digestive tract.

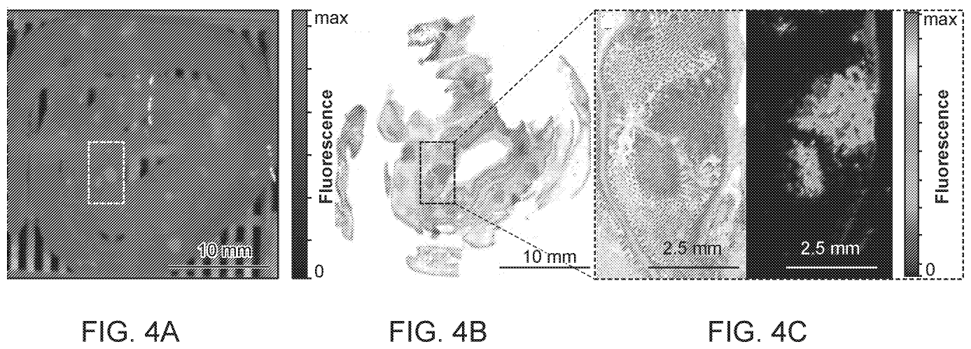

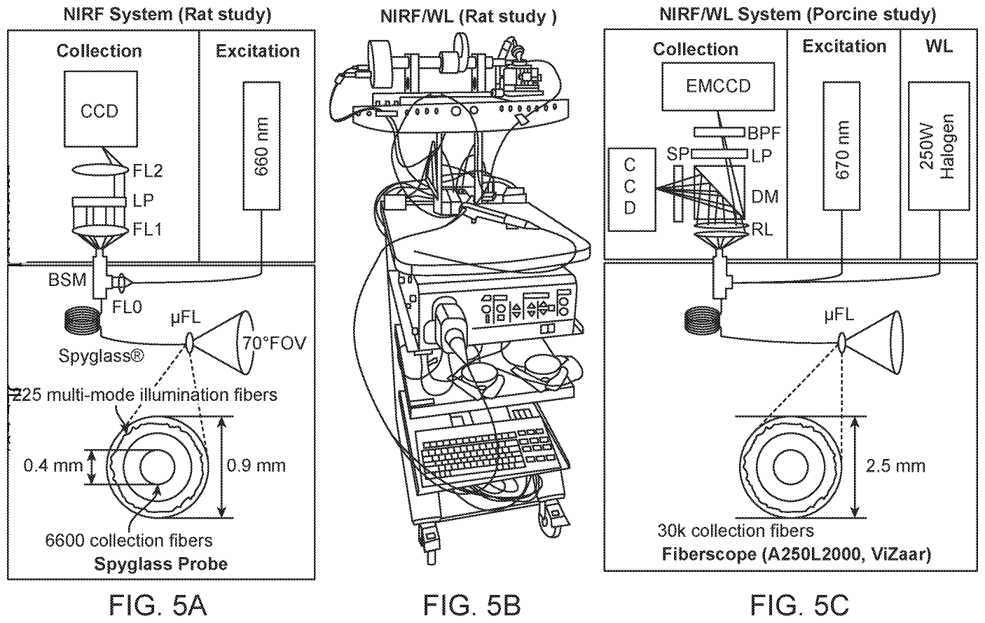

We performed studies in genetically engineered rodent models of gastrointestinal carcinogenesis—the Apc^{Min/+} mouse and Apc^{Pirc/+}. Lesions develop via the Vogelstein sequence, and, as such, the Apc rodent model is a particularly useful model for our purpose of detecting premalignant lesions using FSNs. In studies with the biodegradable FSNs, we have demonstrated that after intravenous administration the FSNs enable highly sensitive detection of dysplastic lesions throughout the intestinal tract of female Apc^{Min/+} mice (n=5; FIG. 4). Furthermore, we demonstrate in the larger male and female Apc^{Pirc/+} (n=5) model that intravenous FSNs successfully enable highly sensitive endoscopic detection when using a dual-modal near infrared fluorescence/white-light (NIRF/WL) endoscopic imaging system (FIG. 5).

The endoscopy results (FIG. 6a) were corroborated by wide-field near-infrared fluorescence imaging on excised colon tissues of the animals that were endoscopically surveilled (FIG. 6b). The first generation FSNs detected both grossly pedunculated polyps as well as sessile dysplastic polyps (FIG. 6c) based on endoscopic and histologic features with tumor-to-background ratios (TBR) of >10, which is significantly higher than the TBR (range 2.2-8.8) of other fluorescent probes that have been evaluated for (endoscopic) adenoma detection. Widespread improvement in the endoscopic recognition of dysplastic GI tract lesions will have important implications for the surveillance and management of incipient GI cancers.

Our use of intravenous near-infrared fluorescent silica nanoparticles as positive contrast agents for endoscopic detection of (pre)malignant lesions of the GI tract is compatible with current clinical practice and instrumentation. An intravenous bolus injection can be administered during the obligate blood-draw procedure prior to endoscopic surveillance. Since the FSNs are fully biodegradable, they can be used routinely in high-risk patients. The high tumor to background (TBR) produced by FSN-augmented fluorescence-assisted endoscopy enables a binary (“yes or no”) readout to reduce interoperator variability, improve (pre)malignant lesion detection and diagnostic accuracy, and enable targeted sampling and resection of visualized lesions. This allows a shift in practice away from the random biopsy technique, where less than 0.1% of the mucosal surface area is blindly sampled, and away from aggressive intervention (e.g. colectomy) for the management of dysplasia in high-risk patients.

As an additional benefit, near-infrared fluorescence (NIRF) imaging offers instant, real-time imaging at a higher resolution and wider field-of-view than Raman imaging, which takes 2-3 h to generate an image.

### Example 2

### Surface Chemistry Modifications and Synthesis Reactions

FSNs are provided with improved pharmacokinetic properties. Reduced off-target uptake by organs of the mononuclear phagocyte system (MPS), such as the liver and spleen, improves nanoparticle bioavailability and leads to enhanced nanoparticle uptake by the tumor. The two major determinants that dictate the biodistribution of nanoparticles are the size and surface chemistry of the nanoparticle. Studies have shown that 50-nm nanoparticles demonstrate superior tumor accumulation, and reduced hepatic uptake relative to larger 100-nm nanoparticles, but previously FSNs have been restricted for clinical use to a minimum size of 10 nm, in order to avoid rapid renal clearance.

The FSN synthesis protocol covalently embeds silane-appended NIRF dyes in the nanoparticle's silica matrix and produces narrowly-dispersed batches of differently-sized FSNs by changing the water concentration (FIG. 2, 11).

Silane-appended NIRF dyes are synthesized either by reacting (3-mercaptopropyl) trimethoxysilane with a commercially-available maleimide functionalized dye (e.g. CF680R-maleimide) or a meso-chloro-substituted near-infrared dye (e.g. IR783) in a solvent (e.g. dimethylsulfoxide) at ambient conditions or 72° C., respectively, for 24 hours. The silane appended NIRF dyes can be used without further purification. For each size, the dye content is optimized to achieve the brightest near-infrared fluorescent signal on a per particle basis (FIG. 2, 11).

The hydrodynamic diameter and physical size of FSNs is characterized using nanoparticle tracking analysis and transmission electron microscopy (TEM), respectively. The effect of 3 different sizes—25, 50, and 100 nm—on the biodistribution and tumor accumulation of equimolar doses FSNs (i.e. same number of nanoparticles for each size) in 12-16 week-old male and female Apc^{Min/+} mice fed a high-fat diet (HFD; n=5, randomly allocated per size) is determined. NIRF imaging is performed 24 h after intravenous administration of equimolar FSN doses (30 fmol/g) on freshly excised tissues using a Pearl Trilogy small animal NIRF imaging (LI-COR, Inc.). The size that produces the highest tumor-to-background and tumor-to liver ratio is selected.

Another critical determinant for biodistribution of nanoparticles is the chemistry at the nanoparticle's surface, which interfaces with the physiological environment. Following intravenous administration, nanoparticles are susceptible to opsonization (i.e. the binding of serum proteins to the nanoparticle's surface via electrostatic or hydrophobic interactions), which activates the complement system to cue the MPS to remove the foreign nanoparticles from the circulation. To extend circulation times and increase passive accumulation at the tumor, polymer coatings (e.g. polyethylene glycol (PEG)) have been applied to reduce opsonization. While PEGylation is a commonly employed strategy, plasma proteins continue to adsorb on the nanoparticle surface even when the nanoparticle surface is decorated with a very dense layer of PEG.

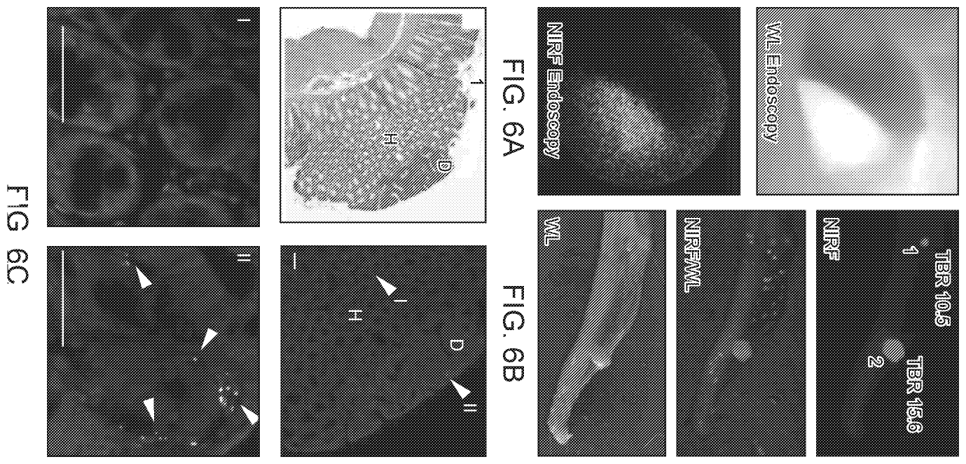

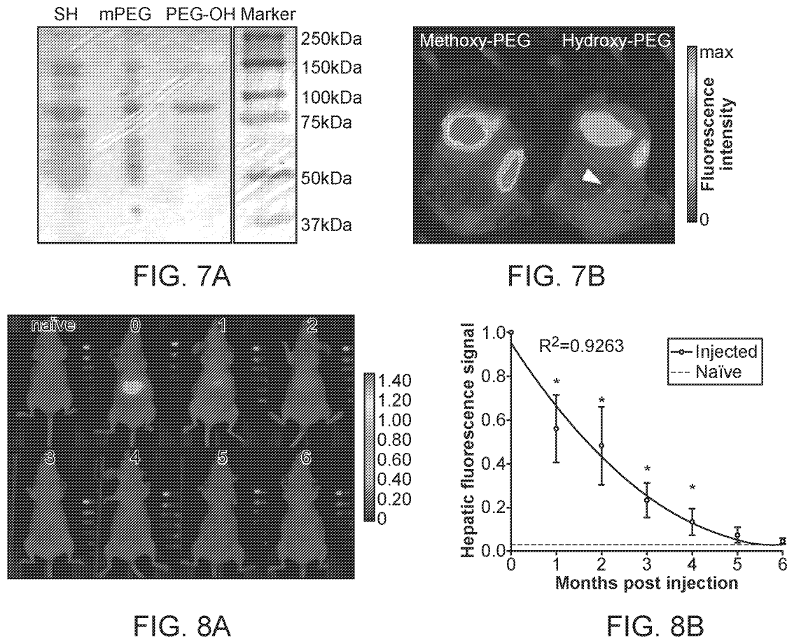

We evaluated a hydroxy-terminated PEG (PEG-OH; Mn 2,000 Da)-based surface coating against the widely used methoxy-terminated PEG (mPEG; Mn 2,000 Da). As shown in FIG. 7, coating of the FSNs with PEG-OH significantly reduced total protein absorption (i.e protein corona) and significantly reduced off-target accumulation in the liver (FIG. 7B). The experiments are repeated to optimize the PEG-length (e.g. Mn 1,000, 2,000, 5,000 Da) in 12-16 week old male and female Apc^{Min/+} mice on HFD (n=5, randomly allocated per length) and functionality (e.g. hydroxy, methoxy, betaine (zwitter-ionic); n=5 randomly allocated per functionality) and the in vitro protein corona formation correlated to the biodistribution of differently functionalized FSNs. To enable scale up of FSN production, tangential flow filtration is used to wash and concentrate the FSNs after synthesis, which allows significant upscaling of FSN production.

The biodegradability of first-generation FSNs was studied and it was found that they fully clear from the liver and spleen. To further limit exposure time, biodegradability can be accelerated by introducing labile bonds within the fluorescent silica matrix, including redox- and acid-labile bonds such as disulfides or esters, respectively, that provide high, short-term stability to enable the detection of (pre)malignant lesions in vivo using NIRF imaging, while reducing MPS exposure time via accelerated biodegradability and subsequent clearance.

Biodegradation kinetics are studied in vitro by incubating the biodegradable FSNs in liver whole tissue lysate and determine the degree of degradation over time using TEM. The in vivo degradation kinetics of optimized FSNs relative to the first-generation FSNs is determined in 8 week-old male and female nude mice (n=5, randomly allocated to first- or next-gen. FSNs). Due to their furless skin, nude mice enable the noninvasive NIRF imaging at multiple time-points to study the biodegradation of FSNs in the liver and spleen in vivo.

### Example 3

FSN are generated to incorporate CF680R (Biotium Inc.), which is a rhodamine-based dye with excitation and emission maxima of 680 and 701 nm, respectively. Due to autofluorescence of food constituents present in the digestive tract, the optical properties of the current “700 nm” FSNs are suboptimal. Furthermore, most clinical endoscopy- and wide-field NIRF imaging systems are only equipped with a 785-nm excitation source and 800-nm long-pass filter for imaging of indocyanine green (ICG), a clinically approved optical contrast agent. “800 nm” FSNs are developed with similar brightness to the “700 nm” FSNs.

Using our established FSN synthesis protocol, clinically-approved dyes are incorporated, including ICG, IRdye800CW, IR783, IR780 and S0456 to select an ‘800 nm’ FSN version that produces the strongest NIRF signal.

### Example 4

### Biodegradability

FSNs upon intravenous administration distribute to MPS organs such as the liver and spleen and are fully cleared from those organs over time. To quantify the actual organ distribution 18 hours after intravenous administration as a percentage of the injected dose (% ID/g), and establish how the FSNs are cleared from the MPS organs (FIG. 8).

### Example 5

### Evaluation of Toxicity

For clinical translation and to support an investigational new drug (IND) application, acute- and long-term toxicity studies are performed to probe the effects of FSN exposure in mice and rats. Since silica-based nanoparticles have already been translated to the clinic and have undergone extensive toxicity-testing no major toxicities are expected.

8-week old C57BL/6J mice and 4-6-month old F344 rats are randomly divided into 3 groups that consist of 24 males and 24 females. Group 1 and 2 receive an intravenous injection of 30 fmol/g or 90 fmol/g, representing the clinical dose and triple the expected clinical dose, respectively. The control group 3 receives an intravenous injection with the vehicle (5% D-glucose in water (D5W)). At day 1, 7, 28, 56, 182, and 404 post injection, animals are sacrificed (4 male/4 female for each group at each time point).

Toxicity assessments include clinical observations and weights, clinical chemistry, including stress hormones (cortical and adrenocorticotropic hormone), hematology, and histopathology of major organs (adrenal gland, aorta, bone with bone marrow, brain, kidney, liver, lung, lymph node, pancreas, prostate, skeletal muscle, spleen, and testis/ovary) by a veterinary pathologist

### Example 6

### Synthesis of Silane-Appended Fluorescent Dye

1 μmol maleimide or NHS-functionalized dye (e.g. CF680R (U.S. Pat. No. 9,579,402)) in 100 μL dry N,N-dimethylformamide (DMF), dimethylsulfoxide (DMSO) or N-methylpyrrolidone (NMP) was reacted with 1 μmol 3-mercaptopropyltrimethoxysilane (MPTMS), 3-mercaptopropyltriethoxysilane (MPTES), 3-aminopropyltrimethoxysilane (APTMS) or 3-aminopropyltriethoxysilane (APTES) overnight at 70° C. in a 1.5 ml container to yield silane-appended dye which was immediately used without any further purification (FIG. 2, 11).

Alternatively, 1 μmol mesochloro-substituted cyanine (e.g. IR780 iodide, IR783, etc), benz[cd]indolium (e.g. IR1048, etc.), or pyrylium (e.g. IR1061, etc.) dye in 100 uL dry DMF, DMSO, or NMP in the presence of triethylamine (TEA) or diisopropylethylamine (DIEA) overnight at 70° C. in a 1.5 mL container to yield the silane-appended dye which was immediately used without any further purification (FIG. 2, 11).

### Example 7

### Synthesis Fluorescent Silica Nanoparticles

A combination of silane-appended dye(s) were added to 2.5 L isopropyl alcohol containing 200 mL water, 50 mL 28% (v/v) ammonium hydroxide, 150 mL silica precursor (e.g. tetraethyl orthosilica) of which a certain fraction consists of a labile-bond containing silica precursor (e.g. bis(triethoxysilylpropyl)disulfide, etc) ranging from 0-50% (v/v). After 15-20 min, the fluorescent silica nanoparticles (FSN) were collected either using centrifugation (10 min; 7500 rpm; 18° C.) or tangential flow filtration (molar weight cut off (MWCO) 100 kDA; modified polyethersulfone mPES filter column).

For surface modification, the FSN were washed with excess ethanol and redispersed in 100 mL ethanol containing 10 mL MPTMS and 5 mL 28% (v/v) ammonium hydroxide. After 2 hours at ambient conditions, the thiol-functionalized FSN were washed with excess ethanol and redispersed in 50 mL water containing 220 mg maleimide-functionalized hydroxy-polyethylene glycol (mal-PEG-OH; M_{w} 3400 da) and allowed to react for at least 2 hours. The hydroxy-PEG functionalized FSN were purified and redispersed in 50 mL injection fluid (5% (w/v) D-glucose in water (D5W)).

### Example 8

Fluorescent silica nanoparticle characterization. The brightest FSNs are obtained when the starting concentration of the silane-appended dye is in the range of 0.1-15 μM. The fluorescence intensity between different batches produced under identical conditions is highly reproducible. Typically, covalent incorporation of texas red-, (lissamine) rhodamine-, or xanthene-based dyes produce FSNs with significantly more stable fluorescence in aqueous environment, unlike covalent incorporation of cyanine-based dyes where a more significant reduction in fluorescence is observed in an aqueous environment relative to alcohols. In FIG. 2, transmission electron graph, hydrodynamic diameter, fluorescence imaging, and the limit of detection of FSNs, which ranges from, but is not limited to, 1 femtomolar (10⁻¹⁵ M)-1 picomolar (10⁻¹² M) on a per particle basis of a typical FSN-PEG-OH is presented. Further, depending on the reaction conditions and the reaction time, FSNs with a size ranging from 25-200 nm can be obtained.

Surface functionality is introduced to FSNs by reacting with functional silanes (e.g. MPTMS, APTES, etc). The introduced functional groups can be used to conjugate polymers or targeting moieties to the FSN surface. A typical FSN preferentially is 100 nm in size and decorated with 10,000-50,000 hydroxy-terminated PEG (PEG-OH) polymers using sulfhydryl-functionality on the FSN surface. Importantly, it should be noted that compared to methoxy-terminated PEG-coated FSNs, hydroxyl-terminated PEG-coated FSNs showed decreased liver uptake.

The FSNs are fully biodegradable within 6 months. This window can be shortened by covalent incorporation of biologically labile bonds (e.g. disulfide, tetrasulfide, esters, amides, cleavable peptides, etc.) within the silica nanoparticle matrix.

### Example 9

### Endoscopic Detection of Dysplasia Using Biodegradable Fluorescent Nanoparticles in Rodent and Porcine Models of Colorectal Carcinogenesis

Early and comprehensive endoscopic detection of colonic dysplasia—the most clinically significant precursor lesion to colorectal adenocarcinoma—provides an opportunity for timely, minimally-invasive intervention to prevent malignant transformation in high-risk patients. To this end, we developed and evaluated the performance of an intravenously (i.v.) administered fully biodegradable near-infrared fluorescent silica nanoparticle (FSN) to specifically high-light dysplastic or malignant colorectal lesions during colonoscopy.

A silane-appended near-infrared fluorescent dye was covalently incorporated into the matrix of a silica nanoparticle using a modified Stöber method (Stöber W, et al. Journal of Colloid and Interface Science (1968)) to yield FSNs with a diameter of 100(±26)-nm. Subsequently, the surface of the FSNs was passivated using hydroxyl-terminated polyethylene glycol (PEG; 3.4 kDa). The biodegradation of i.v. administered PEGylated FSNs (30 fmol/g) was studied in nude mice (n=5), which due to their furless skin enabled longitudinal NIRF imaging of the liver. The ability of FSNs (i.v.; 30 fmol/g) to highlight dysplasia was studied in transgenic rodent- (Apc^{Min/+} mice (n=5); Apc^{Pirc/+} rats (n=8)) and a human-scale model (Apc1311-mutant pig; n=1) 18 hours post-injection on resected colons. Fluorescence-guided endoscopy was performed in anesthetized Apc^{Pirc/+} rats prior to resection of the colons. All resected colons were processed for histopathology and examined by a veterinary pathologist.

The FSNs were shown to be fully biodegradable based on loss of hepatic NIRF signal within 4 months post-injection of FSNs. In addition, no long-term toxicities or adverse events related to intravenous PEGylated FSN administration were observed based on blood chemistry and post-mortem histopathological assessment of major organs. In the Apc^{Min/+} mice, we consistently demonstrated that at the relatively low dose of 30 fmol/g the FSNs highlighted all colorectal lesions. The only observed false positive lesions were Peyer patches—focal lymphatic tissues that are located in the submucosa. In Apc^{Pirc/+} rats FSN-augmented NIRF endoscopy enabled the sensitive detection of colorectal polyps. Subsequent ex vivo wide-field imaging of the resected colons confirmed the findings and showed that FSNs specifically highlight dysplastic colorectal lesions with a signal significantly higher than background (P<0.05) and careful histopathological examination by a veterinary pathologist and confocal near-infrared microscopy corroborated that the nanoparticles specifically accumulate in the stroma of dysplastic lesions. A feasibility study in a Apc1311 mutant pig (n=1) demonstrated that intravenous FSNs enabled specific detection of colorectal dysplasia in a human-scale animal model.

We demonstrate that a fully biodegradable FSN specifically accumulates in dysplastic colorectal lesions (and not in clinically non-relevant hyperplastic lesions) after i.v. administration in transgenic rodent and porcine models of colorectal carcinogenesis. The high tumor-to-background ratios provided by the FSNs enable real-time fluorescent-guided endoscopic surveillance of the colons. Since the FSNs are fully biodegradable and nanoparticles of similar size and composition have been translated to the clinic, we foresee a viable path towards rapid clinical translation.

### Example 10

### Biodegradable Fluorescent Nanoparticles for Endoscopic Detection of Dysplastic Lesions in Animal Models of Colorectal Carcinogenesis

Early and comprehensive endoscopic detection of colonic dysplasia—the most clinically significant precursor lesion to colorectal adenocarcinoma—provides an opportunity for timely, minimally-invasive intervention to prevent malignant transformation. Here, we describe the development and evaluation of biodegradable near-infrared fluorescent silica nanoparticles (FSN) to highlight dysplastic lesions and improve adenoma detection during fluorescence-assisted white-light colonoscopic surveillance in rodent and human-scale models of colorectal carcinogenesis. We demonstrate that the FSNs are biodegradable (t_{1/2} of 2.7 weeks), well-tolerated, and enabled detection and delineation of dysplastic colorectal lesions as small as 0.5 mm² with high tumor-to-background ratios. Furthermore, in the human-scale, APC^{1311/+} porcine model, we demonstrated the clinical feasibility and benefit of using FSN-guided detection of dysplastic colorectal lesions using video-rate fluorescence-assisted white-light endoscopy. Since nanoparticles of similar size (e.g. 100-150-nm) or composition (i.e. silica, silica/gold hybrid) have already been successfully translated to the clinic, and, clinical fluorescent/white light endoscopy systems are becoming more readily available, there is a viable path towards clinical translation of this strategy for early colorectal cancer detection, and prevention in high-risk patients.

Conventional white-light (WL) endoscopy plays a key role in the detection and removal of lesions of the digestive tract. In fact, early endoscopic detection and removal of (asymptomatic) colorectal dysplasia—the main precursor lesions of gastrointestinal (GI) cancers—significantly reduces cancer risk and its associated death by 83 and 89%, respectively. However, a substantial miss rate has been reported for WL detection of (pre)malignant lesions particularly in high risk patients (e.g. inflammatory bowel disease, Lynch syndrome, etc.), which compromises early detection and intervention. This significantly increases the risk of (interval) cancer and its associated mortality. Important reasons for the miss rate are their subtle appearance that may appear nonpolypoid (flat or depressed), or lesions are located behind folds, or are not endoscopically identifiable altogether. Moreover, the subtle appearance complicates determination of the true lateral extent, thus impedes the ability to achieve complete endoscopic mucosal resection of these lesions resulting in recurrence rates of 15-26%. Targeted biopsy using topically-applied dyes to delineate mucosal abnormalities (i.e. chromoendoscopy) has been shown to improve the adenoma detection rate by 30%. However, chromoendoscopy is not embraced by endoscopists due to the perceived hassle, cost, and time associated with intraluminal dye administration, and digital (image-enhanced) chromoendoscopy (e.g. narrow-band imaging (NBI), Fuji Intelligent ChromoEndoscopy (FICE)) have only shown marginally improved adenoma detection rates.

To mitigate the high miss-rate and low diagnostic accuracy of conventional white-light endoscopy, and, negate the perceived drawbacks of chromoendoscopy, we improved early detection of (incipient) colorectal cancer during endoscopic surveillance using nanoparticle-based optical contrast agents. The rationale was based on our observation that systemically administrated 100-nm Raman nanoparticles passively accumulated in a wide variety of tumors including colorectal adenomas. However, gold-based Raman nanoparticles are non-biodegradable and display long-term sequestration by the liver and spleen (>5 months) after intravenous administration. This can limit periodic, intravenous clinical application of these non-biodegradable Raman nanoparticles for routine screening in high-risk patients. To benefit from the inherit tumoritropic properties of nanoparticles and address the issue of long-term sequestration, we developed biocompatible and biodegradable ‘nanosimilar’ near-infrared fluorescent silica nanoparticles (FSN). Evaluation in animal models of colorectal carcinogenesis demonstrated that FSNs are biodegradable, well-tolerated, and, enable real-time detection of dysplastic colorectal lesions using near-infrared fluorescence-assisted white-light endoscopy (NIRF; here defined as excitation, emission >650 nm) in transgenic rodent- and human-scale, porcine models of colorectal carcinogenesis. Furthermore, the FSNs were designed to have optical properties that are fully compatible with existing clinical NIRF/WL endoscopy systems to facilitate clinical translation.

### Results

Synthesis and characterization of fluorescent silica nanoparticles (FSN). Biodegradable fluorescent silica nanoparticles were synthesized using a modified Stöber reaction in the presence of a (3-mercaptopropyl)trimethoxysilane-appended near-infrared dye (CF680R-MPTMS) to ensure covalent incorporation of the dye into the silica nanoparticle matrix (FIG. 2). A CF680R-MPTMS concentration of 0.75 μM proved to be optimal and reproducibly yielded FSNs with the highest fluorescence intensity on a per particle basis (FIG. 2, 11, 12). The surface of the FSNs was modified using MPTMS to introduce thiol-functionality, which, in turn, were used for passivation of the FSN surface with hydroxy-terminated polyethylene glycol (PEG-OH; 3.4 kDa) via straightforward maleimide chemistry. The zeta potential of the bare and PEGylated fluorescent silica nanoparticles (FSN)s was measured to be −41.8±6.3 mV and −20.2 ±7.0 mV, respectively. The silica core of the PEGylated FSNs had a size of 100 nm as determined by transmission electron microscopy (TEM). Following PEGylation with hydroxy-terminated PEG (3.4 kDa), the hydrodynamic diameter of the FSNs increased by 17 nm. The Flory radius of ˜8.5 nm indicate that the PEG chains had assumed a brush-confirmation, which has been shown to be significantly more resistant to plasma protein adsorption. In fact, when incubated for 1 h in human serum, less protein had adsorbed to the FSN (PEG-OH grafted) versus bare, fluorescent silica nanoparticle cores (FIG. 7). The FSNs were shown to have a limit of detection of 10 femtomolar (10×10⁻¹⁵ M), which was an order-of-magnitude higher than previously described Raman nanoparticles with a non-biodegradable gold nanocore (Harmsen S, et al. Science Translational Medicine (2015)).

In vivo biodegradability of non-PEGylated FSNs and FSNs. To determine the biodegradation kinetics of the FSNs, we injected either non-PEGylated FSN, FSNs at equimolar amounts, or vehicle control intravenously (i.v.) into nude mice (n=5/group). The administrated FSN dose of 30 fmol/g was equivalent to the dose of Raman nanoparticles that was used in our previous study. The study was performed in nude mice to allow longitudinal monitoring of hepatic near-infrared fluorescence in the same animals over a 6-month time period post systemic FSN administration. As shown in FIG. 8, 1-d (‘0’) post injection of an equimolar dose of fluorescent nanoparticles, the biodistribution of the non-PEGylated FSNs versus FSNs was distinct. While the non-PEGylated demonstrated high hepatic uptake, FSNs distributed to the liver and spleen. Based on the loss of significance between the hepatic fluorescence intensity of the injected animals compared to the background fluorescence in naïve animals (FIG. 8b), it was concluded that the FSNs and non-PEGylated FSNs fully degrade and clear within 3-months (t_{1/2} 2.7 weeks) and 4-months (t_{1/2} 3.0 weeks) post injection, respectively. TEM demonstrated a biodegradation pattern of the FSNs in situ that was similar to that observed in vitro with the FSNs etching from the inside, collapsing, and dissolving (FIG. 13).

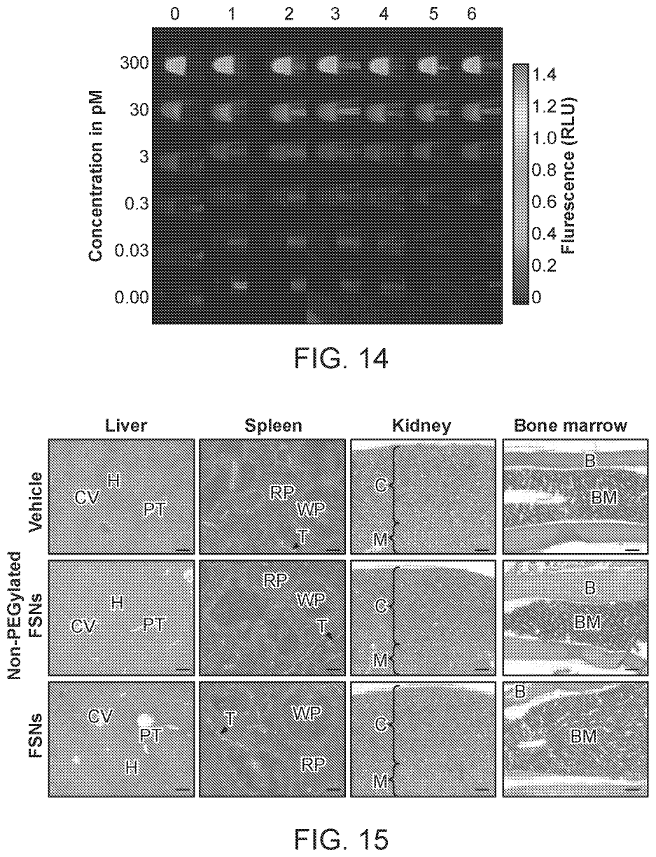

To ensure that the decrease in fluorescence signals was due to FSN biodegradation and clearance, and not due to photobleaching resulting from the repeated imaging, we included a standard that contained a dilution series of the exact same batch of fluorescent silica nanoparticles as the ones that were injected, was included. The coefficient of variance (CV) of the near-infrared fluorescent signal was <5% over the 6 months period (˜200 exposures) indicating the fluorescent silica nanoparticles should be photostable and the decrease in hepatic fluorescence signal should not be due to photobleaching (FIG. 14). Throughout the biodegradation study, the animals were closely monitored, and no overt signs of pain or distress were observed. Following the biodegradation study, all animals were humanely euthanized, and major organs (i.e. liver, spleen, kidney) were harvested and submitted for histopathological assessment. No abnormalities were found in the inspected organs after long-term exposure to the FSNs indicating that the non-PEGylated FSNs as well as the PEGylated FSNs were well-tolerated at the current dose (FIG. 15).

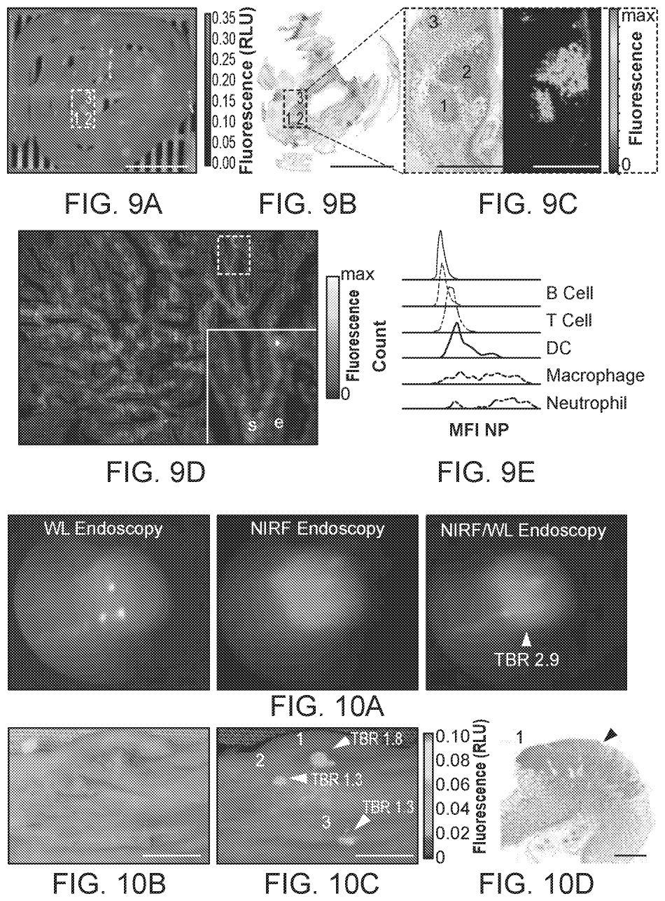

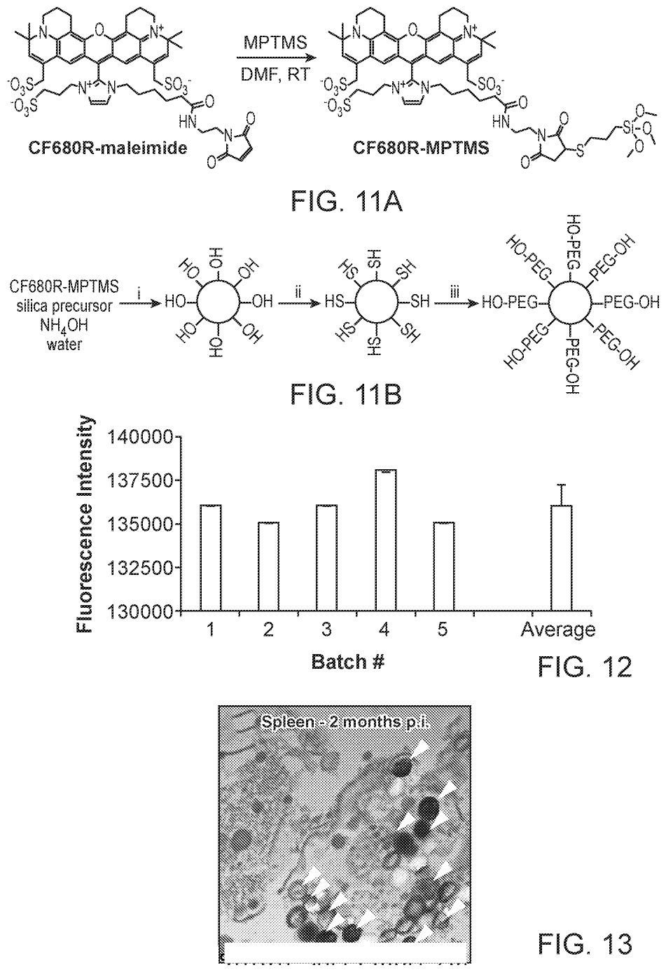

Detection of adenomas in the intestinal tracts of Apc^{Min/+} mice. We used the Apc^{Min/+} mouse model of familial intestinal carcinogenesis to determine the ability of FSNs to provide detection and visualization of intestinal adenomas. The Apc^{Min/+} mice (n=5) received 75 μl of 10 nM FSNs in 5% (w/v) D-glucose in water (D5W) to achieve a dose of 30 fmol/g via i.v. injection. Since the Apc^{Min/+} mice most commonly develop adenomas in the small intestine, (18, 19) the intestinal tracts of the injected animals were harvested and imaged 1-day post injection. As shown in FIGS. 4 and 5 FSNs enables instant, wide-field near-infrared fluorescence (NIRF) imaging of freshly resected ileal tissues. The tumor-to-background ratio (TBR) of selected polyps was >3 and polyps as small as 0.5 mm2 (lesion 3) were detected. Histopathological assessment of the tissues identified the FSN-positive lesions as adenomatous polyps. Since hematoxylin and eosin do not fluoresce >700 nm, we performed NIRF imaging of the H&E-stained tissue sections and validated the specificity of the FSNs for the adenomatous polyps after intravenous administration in ApcMin/+ mice (FIG. 9d).

To probe the intratumoral fate of the intravenously injected FSNs, we performed high magnification NIRF confocal microscopic imaging on the H&E stained tissue section of the adenoma. It was shown that upon extravasation, the FSNs specifically localize to and reside within the stromal compartment of the adenoma and do not readily interact with the epithelial cells lining the adenoma. Furthermore, within the tumor stroma the FSNs were found to be mostly associated with neutrophils and tumor-associated macrophages (FIG. 9e).

FSN-augmented endoscopic detection of dysplastic colorectal lesions in Apc^{Pirc/+} rats. To assess the ability of FSNs to highlight colorectal adenomas in a preclinical endoscopic scenario, we intravenously administered FSN (30 fmol/g) to Apc^{Pirc/+} rats (n=5) 18 hours prior to endoscopy. In contrast to the Apc^{Min/+} mouse model, Apc^{Pirc/+} rats predominantly develop adenomas and localized adenocarcinomas in the colon in a similar manner as human patients with familial adenomatous polyposis (FAP) or sporadic colorectal cancer. Furthermore, the larger body size of the rats enables the accommodation of the endoscope to perform colonic surveillance. The endoscopy system constitutes a clinical white-light endoscope that is equipped through its working-channel with an FDA-cleared Spyglass fiberoptic probe (FIG. 5).

Combined NIRF/WL endoscopy was performed in Apc^{Pirc/+} rats that had received i.v. doses of FSNs (30 fmol/g) 18-hours (h) prior to endoscopy. As shown in FIG. 6a, FSNs highlighted adenomas during NIRF/WL endoscopic surveillance of the colon in these animals. Of note, the signal produced by the FSNs was sufficiently high to enable real-time endoscopic surveillance at a frame-rate of at least 5.0 fps. To validate the NIRF/WL endoscopy findings, the colons were collected, dissected, and imaged on a wide-field NIRF imaging system immediately after endoscopic surveillance. The wide-field NIRF imaging supported the NIRF/WL endoscopy findings and demonstrated that the FSNs selectively accumulated in colorectal adenomas (TBRs >10), which was confirmed after histopathological assessment. Interestingly, upon closer inspection of the H&E stained tissue section of the smaller adenoma (‘1’), it was shown to have a mixed morphology of a hyperplastic polyp and adenoma with an isolated dysplastic focus. NIRF microscopy of the mixed hyperplastic adenomatous polyp demonstrated the specific accumulation of FSNs in the stromal compartment of the dysplastic focus and not in the surrounding hyperplastic and normal colorectal tissues.

FSN-augmented endoscopic detection of dysplastic colorectal lesions in the human-scale APC^{1331/+} porcine model of colorectal carcinogenesis. FSNs are not yet approved in humans for colorectal dysplasia detection. To assess clinical feasibility of our approach, we performed a large animal study in a human-scale model of colorectal carcinogenesis—the APC^{1331/+} porcine model. APC^{1331/+} pigs carry a gene mutation orthologous to a common germline mutation found in human FAP patients (i.e. APC1309) and develop high-grade dysplastic colorectal adenomas. Based on allometric scaling of the rodent dose (30 fmol/g), a dose of ˜5 fmol/g was selected for administration to the APC^{1331/+} pigs. Accordingly, two APC^{1331/+} pigs weighing 79 and 94 kgs received an intravenous injection of 15 ml 25 nM FSNs in D5W to achieve a dose of 4.7 and 4.0 fmol/g, respectively. The next day, the colons of the anesthetized animals were surveilled using combined NIRF/WL endoscopy (FIG. 5; FIG. 10). At the selected dose, the FSNs highlighted the adenomas and enabled real-time, combined NIRF/WL surveillance at a frame-rate of at least 5.0 fps. Following endoscopy, one animal (with weights 94) was sacrificed and its colons was harvested, and formalin fixed. The fixed tissue was subjected to wide-field NIRF imaging (FIG. 10). The adenomatous polyps were highlighted by the FSNs during wide-field NIRF imaging and demonstrated TBRs of >1.3. Histopathological assessment of the FSN-positive lesions proved the lesions were adenomatous polyps with dysplastic foci.

We developed biodegradable fluorescent silica nanoparticles (FSN) that are well-tolerated and highlight dysplastic colorectal lesions—specifically dysplastic lesions; the most clinically significant precursor lesions to colorectal adenocarcinoma—during video-rate, near-infrared fluorescence-assisted white-light colonoscopic surveillance in small- and human-scale animal models of colorectal carcinogenesis. The presented advantages of our FSN-based strategy to highlight adenomas during fluorescence-assisted white-light colonoscopy are aimed at improving the miss rate of colonoscopy, and to address the perceived hassle, cost, and time associated with intraluminal dye administration for chromoendoscopy.

In recent years, several targeted, molecular imaging strategies have been investigated highlighting colorectal lesions during fluorescence-assisted white-light endoscopy. Most notably, vascular endothelial growth factor (VEGF) or epidermal growth factor receptor (EGFR) targeting antibodies or c-MET targeting peptides labeled with NIRF dyes to minimize tissue autofluorescence, have shown great promise in improving adenoma detection during fluorescence-assisted white-light colonoscopy in the clinic. However, often active targeting approaches are limited by target (over)expression and heterogeneity, specificity, and accessibility at the tumor site. For instance, EGFR is overexpressed in 50% of colorectal adenomas and heterogeneously expressed in those positive lesions. Moreover, since target expression may not be tumor stage-specific it may lead to over-diagnosing, as illustrated in a clinical trial with an intravenous c-Met targeting probe that not only highlighted colorectal adenomas, but also hyperplastic polyps, which have no clinical relevance.

In contrast to active targeting approaches, passive tumor targeting using fluorescent dye—embedded nanoparticles (>10 nm) obviates the need for specific targeting moieties, because tumor accumulation is governed by a biologically phenomenon that is shared by lesions across the cancer spectrum ranging from dysplastic—to advanced malignant diseases; the enhanced permeability and retention (EPR) effect. The enhanced permeability of the tumor neovasculature facilitates extravasation of nanoparticles into the tumor bed where they are locally retained due to ineffective lymphatic drainage. Since it has been shown that EPR strongly correlates with the degree of tumor vascularization and dysplastic colorectal lesions commonly have increased vascularity, nanoparticles such as Raman nanoparticles and FSNs can specifically accumulate in clinically relevant dysplastic lesions.

Unlike the Raman nanoparticles, however, FSNs consist solely of dye-embedded silica—a biocompatible and biodegradable material that has already been translated to the clinic. In addition, FSNs have a detection limit that is only one order-of-magnitude poorer than that of Raman nanoparticles, which to date have showcased the lowest reported limit of detection using (near) real-time optical imaging. Relative to other fluorescent-based agents such as free- or targeted dyes (e.g. indocyanine green (ICG), IRdye800CW, respectively) or liposomal dye formulations, which typically have a limit of detection in the picomolar range (10-12 M), FSNs have a limit of detection in the low femtomolar range (10-14 M; FIG. 2g). Covalently-incorporated dyes within the silica matrix exhibit photophysical properties that are distinct from their solution properties, thereby leading to enhanced radiative emission and increased photostability.

Our studies were performed in genetically engineered rodent and human-scale, porcine models of gastrointestinal carcinogenesis—the Apc^{Min/+} mouse, Apc^{Pirc/+} rat, and APC^{1331/+} pig. The rodent models have been criticized for not (or rarely) progressing to invasive carcinoma. However, in all species the lesions develop via the Vogelstein-sequence, and, as such, the Apc-driven carcinogenesis animal models are particularly useful for our purpose of evaluating endoscopic detection of dysplastic lesions using FSNs. In fact, we demonstrated that after intravenous administration the FSNs enable detection of dysplastic lesions as small as 0.5 mm² throughout the intestinal tract of Apc^{Min/+} mice and in the larger Apc^{Pirc/+} rat- and APC^{1311/+} porcine model. FSNs were found to accumulate in both grossly pedunculated polyps as well as sessile dysplastic polyps.

Widespread improvement in the endoscopic recognition of dysplastic colorectal lesions will have important implications for the surveillance and management of incipient colorectal cancers and cancer prevention. Our proposed use of intravenous FSNs as positive contrast agents for endoscopic detection of (pre)malignant lesions of the GI tract is fully compatible with current clinical practice and instrumentation. For instance, an intravenous bolus injection can be administered during the obligate blood-draw procedure prior to endoscopic surveillance. Furthermore, since the FSNs are fully biodegradable, they can be used routinely in high-risk patients. Lastly, the TBRs produced by FSN-augmented fluorescence-assisted endoscopy enables a binary (“yes or no”) read-out to reduce interoperator variability, improve (pre)malignant lesion detection and diagnostic accuracy, and enable targeted sampling and resection of visualized lesions to allow a shift in practice away from the random biopsy technique, where less than 0.1% of the mucosal surface area is blindly sampled, and away from aggressive intervention (e.g. colectomy) for the management of dysplasia in high-risk patients.

In conclusion, we have developed a biodegradable fluorescent nanoparticle that highlights dysplastic adenomas in animal models of colorectal carcinogenesis. With clinical translation in mind, future studies will be aimed at dose de-escalation and long-term toxicity risk assessment. The FSN dose that was used throughout the reported study was based on the Raman nanoparticle dose as reported in a previous study. Therefore, to find the minimum effective dose, FSN dose de-escalation studies are performed the lowest dose at which adenomas are still detectable using FSN-enhanced NIRF/WL colonoscopy determined. Early colorectal disease detection in high-risk patients, who undergo routine (e.g. annual) screening to monitor disease development or progression, is targeted. The effect of FSN dose accumulation on organs of the mononuclear phagocyte system (e.g. liver, spleen, bone marrow), which avidly take up FSNs following intravenous administration is determined.

### Methods

Materials. All chemicals were purchased from Sigma-Aldrich (St. Louis, Mo.) unless stated otherwise, were of the highest purity available, and used without any further purification.

Fluorescent silica nanoparticle (FSN) synthesis. CF680R-maleimide (1 μmol in 100 μl dry N,N-dimethylformamide (DMF); Biotium Inc., Fremont, Calif.) was reacted with (3-mercaptopropyl)trimethoxysilane (MPTMS; 2 μmol to yield silane-appended CF680R (CF680R-MPTMS), which was used without any further purification. Typically, CF680R-MPTMS (4 μl 10 mM in DMF) was added to 50 ml 2-propanol containing 3.5 ml water, 1.5 ml 28% (v/v) ammonium hydroxide, and 2.5 ml tetraethyl orthosilicate (TEOS). After 15 min, the fluorescent silica nanoparticles were collected by centrifugation (10 min; 7,500 g; 20° C.), washed with excess ethanol, and redispersed in 2.5 ml ethanol containing 50 μl 28% (v/v) ammonium hydroxide and 150 μl MPTMS. After 90 min at ambient conditions, the thiol-functionalized FSN were washed with excess ethanol. The thiol-functionalized FSNs were stored in ethanol at 4° C. On the day of injection, the thiol-functionalized FSNs were collected by centrifugation and redispersed in 2 mL 10 mM 3-(N-morpholino)propanesulfonic acid buffer (MOPS; pH 7.3) containing 3.5 mg maleimide-functionalized hydroxyl-terminated polyethylene glycol (PEG-OH; Mw 3,400 da; Creative PEGWorks, Chapel Hill, N.C.) and allowed to react for at least 2-h at ambient conditions. The PEG-OH functionalized fluorescent silica nanoparticles (FSN)s were purified and redispersed in 1.0 mL 22-μm filter-sterilized 5% D-glucose (D5W) at a concentration of 10 nM.

FSN characterization. FSN size/integrity, hydrodynamic diameter/concentration, and limit of detection were characterized using transmission electron microscopy (TEM), nanoparticle tracking analysis (NTA), and near-infrared fluorescence (NIRF) imaging, respectively. In brief, 1 μl of an FSN dispersion was pipetted onto a carbon-coated grid (CF300-Cu, Electron Microscopy Sciences), air-dried, and loaded into an JEOL 1200ex-II transmission electron microscope operating at 80 kV. The hydrodynamic diameter and concentration of FSNs were determined using NTA using a 1000-fold diluted sample of an FSN dispersion in water. The limit of detection of FSNs was determined by imaging a concentration series of FSNs (3-fold dilution factor) on a Pearl Trilogy NIRF imaging system (LI-COR Biosciences, Lincoln, Nebr.). The zeta potential of 5.0 nM dispersion of non-PEGylated and PEGylated FSNs in 0.22-μm filtered 20 mM MOPS (pH 7.3) was measured using a Zetasizer Nano ZS (Malvern). Biodegradation of FSN was verified in vitro. FSNs (1.0 nM) were incubated in 2504 50% human serum at 37° C. At days 0, 3, 6, and 9, 50 μl was sampled, washed with excess water, collected using centrifugation (10,000 g), and analyzed using TEM.

In vivo study. The in vivo studies at Stanford University (i.e. mouse and rat) were conducted under an Institutional Animal Care and Use Committees (IACUC)-approved protocols and animals were under the direct oversight of an animal care and use program that was AAALAC International-accredited and PHS-assured. The in vivo studies in the porcine model were performed at the Technical University of Munich and were approved by the Federal Government of Bavaria. All applicable institutional guidelines for the care and use of animals were followed. Athymic nude mice (Charles River Laboratories, Wilmington, Mass.), Apc^{Min/+} mice (Jackson Laboratory, Bar Harbor, Me.), and Apc^{Pirc/+} rats (Rat Resource & Research Center, Columbia, Mo.) were fed Teklad Global 2018 diet (Envigo, Huntingdon, UK), which contains 18% protein, 6% fat, moderate phytoestrogens and no alfalfa. APC1311/+ pigs were fed a normal diet.

Biodegradability and biocompatibility. Non-PEGylated, thiol-functionalized fluorescent silica nanoparticles or FSNs grafted with PEG-OH (3.4 kDa) in D5W were intravenously administered at a dose of 30 fmol/g to 2-month old, female nude mice (n=5/group). A separate group of 2-month old, female nude mice (n=5/group) received an intravenous injection of the vehicle D5W. After 24-h, the animals were imaged (t=‘0’) on a small animal NIRF imaging system (Pearl Trilogy, LI-COR Biotechnology, Lincoln, Nebr.). Following monthly imaging for 6 months, the animals were euthanized by CO2 asphyxiation and cardiac exsanguination. Select tissues (liver, spleen, kidney, and bone marrow) were harvested and immersion-fixed in 10% neutral-buffered formalin for 72-h. Femurs were harvested for bone marrow analysis and immersion-fixed/decalcified in Cal-Ex II Fixative/Decalcifier (Fisher Scientific, Fair Lawn, N.J., USA) for 72 hours. Formalin-fixed tissues were processed routinely, embedded in paraffin, sectioned at 5 μm, and stained with hematoxylin and eosin (H&E). H&E sections were blindly evaluated by a board-certified veterinary pathologist (KMC) for treatment-related toxicity. Of note, a small section of the liver and spleen of selected animals was fixed in electron microscopy fixative (2% glutaraldehyde, 4% paraformaldehyde in 0.1 M sodium cacodylate; pH 7.4) and submitted to Stanford Microscopy Facility for transmission electron microscopy analysis. Tissue sections were counterstained and imaged on a JEOL JEM-1400 operating at 120 kV.

Detection of adenomas in Apc^{Min/+} mice. Conscious female Apc^{Min/+} mice (14-20-week old; n=5) received intravenous injections of FSNs (30 fmol/g) via the tail vein using a tail-restrainer. The next day, the animals were deeply anesthetized using inhalant isoflurane (Forane, Baxter, Deerfield, Ill.) and then euthanized via cervical dislocation. The intestinal tissues of the animals were immediately collected, rinsed with PBS and immediately imaged on a Pearl Trilogy NIRF imaging system. Upon completion of imaging, the intestinal tissues were immersion-fixed in 10% neutral-buffered formalin for 24-h and processed for paraffin-embedding and H&E staining. H&E stained tissue section (5- and 10-μm thickness) were re-imaged on an Odyssey NIRF imaging system (Pearl Trilogy, LI-COR Biotechnology, Lincoln, Nebr.) and BZ-X700 NIRF microscope (Keyence, Itasca, Ill.).

Fluorescence-activated cell sorting (FACS) of FSN-associated cells within polyps. Conscious female Apc^{Min/+} mice (18-week old; n=2) received intravenous injections of FSNs (30 fmol/g) via the tail vein using a tail-restrainer. The next day, the animals were deeply anesthetized using inhalant isofluorane and then euthanized by cervical dislocation. Small intestinal tissue containing adenomas was collected and sectioned into small (2-3 mm) pieces and then placed in a dounce glass homogenizer to create a cell suspension. Cells were passed through a 40-μm filter with Hank's balanced salt solution (HBSS) containing DNAse. Cells were counted and resuspended in PBS at a concentration of 1″ 106 cells, prior to live dead staining with fixable LD aqua (#L34957, ThermoFisher Scientific, Waltham, Mass.) for 15 min. Cells were then washed and resuspended in PBS containing 2% bovine serum albumin (BSA), prior to staining with the fluorophore conjugated antibody panel (Pacific Blue anti-human CD11c (1:40; 117321), R-phycoerythrin (PE)-Cy7 anti-human Ly-6G (1:40; 127618), Allophycocyanin (APC)-Cy7 anti-human CD11b (1:40; 101225), PE anti-human CD3 (1:40; 100206), Peridinin-Chlorophyll protein (PerCP)-Cy5.5 anti-human CD45 (1:40; 103132); Biolegend, San Diego, Calif.) for 45 min. Cells were then washed and resuspended in 200 μl of PBS and then analyzed on a BD LSRII flow cytometer. All samples were analyzed in triplicate.

Endoscopic detection of adenomas in Apc^{Pirc/+} rats. Conscious six-month old female and male Apc^{Pirc/+} rats (n=5) received intravenous injections of FSNs (30 fmol/g) via the tail vein using a tail-restrainer. The next day, the animals were anesthetized using inhalant isoflurane (Forane, Baxter, Deerfield, Ill.). The colons of the anesthetized animals were lavaged with phosphate-buffered saline (PBS). Endoscopy was performed with our custom-built combined NIRF/white-light (WL)/imaging endoscopy system equipped with Spyglass fiberscope (Boston Scientific, Marlborough, Mass.), a 660-nm excitation laser operating at 10 mW (IBeamSmart, PT 70-75 mW, Toptica Photonics AG, Gräfelfing, Germany), 664-nm long-pass filter (RazorEdge, LP02-664RU-25, Semrock, Rochester, N.Y.), and an electron multiplying charge-coupled device (EMCCD) camera (Luca R, Andor Technology, Belfast, UK). For a detailed description of the combined NIRF/WL endoscopy systems, please see FIG. 5. The colons of the animals were surveilled and WL and NIRF imaging were concomitantly recorded using Captivate screen-capturing software (Adobe Inc, San Jose, Calif.). No video post-processing was performed with the exception of occasional adjustment of contrast- and brightness levels (applied to the full image). Following endoscopy, the animals were deeply anesthetized using inhalant isoflurane (Forane, Baxter, Deerfield, Ill.) and then euthanized via cervical dislocation. The intestinal tissues of the animals were immediately collected, rinsed with PBS and immediately imaged on a Pearl Trilogy NIRF imaging system. Upon completion of imaging (less than 10 minutes), the colonic tissues were immersion-fixed in 10% neutral-buffered formalin for 24 hours and processed for paraffin-embedding and H&E staining. H&E stained tissue sections (5-μm thickness) were imaged using a custom set-up inverted digital fluorescence microscope (DM6B Leica Biosystems, Buffalo Grove, Ill.) equipped with a highly sensitive Leica DFC9000GTIs camera (4.2M Pixel sCMOS camera), a Cy5.5 filter cube (49022-ET-Cy5.5; Chroma Technology Corp., Bellows Falls, Vt.), and a xenon arc lamp LB-LS/30 (Sutter Instrument) for NIRF imaging of FSNs. Image acquisition and processing were performed using LAS X software (Leica Biosystems).

Endoscopic detection of adenomas in APC^{1311/+} pigs. All experiments involving the APC^{1311/+} pigs were performed in Germany. Sedated male APC^{1311/+} pigs (18-21 months old; n=2) were injected intravenously with the FSNs (30 pmol/kg) via a preplaced catheter into ear vein. The next day, the pigs were anesthetized by intramuscular (i.m.). injection of ketamine (20 mg/kg body weight) and azaperone (2 mg/kg body weight) and fluorescence-guided endoscopy was performed using a custom-built combined NIRF/WL endoscopy system equipped with a 670-nm laser and a ViZaar fiberscope (A250L2000; For detailed information see FIG. 5). The animals that were euthanized were first sedated by i.m. administration of ketamine (20 mg/kg body weight) and azaperone (2 mg/kg body weight), rendered unconscious by a nonpenetrating captive bolt gun applied to the forehead and then immediately exsanguinated. Liver, spleen, kidney, colorectal tissues and muscle were harvested. The freshly resected colorectal tissues were randomly cut in sections of approximately 15 cm in length. All tissues were fixed in 10% neutral-buffered formalin. The fixed tissue sections were imaged on a Pearl Trilogy NIRF imaging system and processed for paraffin-embedding and H&E staining.

Statistical analysis. To calculate the tumor to background ratio (TBR), regions of interest (ROI) were drawn tightly around the tumor and on the tissue background. TBR=ROItumor/ROItissue background. Statistical analysis was performed in Excel (Microsoft). Detailed information on the sample size is described in the figure legends. All values in figures are presented as means±SD unless otherwise noted in the text and figure legends. Statistical significance was calculated on the basis of the Student's t-test (two-tailed, unpaired), and the level of significance was set at least P values <0.05.

1. A biodegradable fluorescent silica nanoparticle (FSN) for in vivo imaging, wherein the FSN is of from about 25 nm to about 200 nm in diameter, comprised of:
(a) a dye that fluoresces in the near infrared spectrum which is (i) covalently joined to a silane, and (ii) distributed throughout the nanoparticle; and
(b) silica distributed throughout the nanoparticle.

2. The FSN of claim 1, wherein the FSN is coated with one or more of hydroxy-terminated polyethylene glycol (PEG); methoxy-terminated polyethylene glycol (PEG);
and targeting moiety-terminated PEG.

3-4. (canceled)

5. The FSN of claim 2, wherein the PEG is covalently linked to the surface of the FSN by sulfhydryl bonds.

6. The FSN of claim 2, wherein the PEG is from about 250 da to about 10,000 da in size.

7. The FSN of claim 2, wherein an FSN comprises from about 10⁴ to about 10⁶ PEG moieties.

8. The FSN of claim 1, wherein the dye fluoresces in the NIR I window at a wavelength of from about 680 nm to about 900 nm.

9. The FSN of claim 1, wherein the dye fluoresces in the NIR II window at a wavelength of from about 900 nm to about 2500 nm.

10. The FSN of claim 1 wherein the detection limit is from about 30×10^{-‥}M to about 10⁻¹³ M on a per particle basis.

11. The FSN of claim 1 wherein the FSN is fully biodegraded after about 3 to about 6 months following administration.

12. The FSN of claim 1 wherein the FSN is fully biodegraded after about 1 to about 6 months following administration due to incorporation of labile bonds.

13. A pharmaceutical composition, comprising:
an FSN of claim 1; and
a pharmaceutically acceptable excipient.

14. A method of detecting dysplastic lesions in the gastrointestinal tract, the method comprising:
administering a pharmaceutical composition of claim 13 to an individual; and following a period of time sufficient for enhanced permeability and retention effect (ERR) to concentrate the FSN at cancerous or pre-cancerous lesions; detecting the presence of the FSN, wherein increased concentration relative to normal tissue of the FSN is indicative of a premalignant or malignant lesion.

15. The method of claim 14, wherein the FSN is administered intravenously, intraluminally, or topically.

16-17. (canceled)

18. The method of claim 14, wherein the period of time sufficient to concentrate the FSN is from about 15 minutes to about 24 hours.

19. The method of claim 14, wherein detection is accomplished with a NIRF or combined NIRF and white light camera.

20. The method of claim 14, wherein detection is used to guide endoscopic surveillance.

21. The method of claim 14, wherein detection is used to guide endoscopic intervention or biopsy.

22. The method of claim 14, wherein detection is used to guide laparoscopic intervention

23. The method of claim 14, wherein detection of the FSN is used to guide surgery of the lesion.
